(19) United States
(12) Patent Application Publication
(10) Pub. No.: US 2016/0237487 A1
(43) Pub. Date: Aug. 18, 2016

(54) Modeling and Predicting Differential Alternative Splicing Events and Applications Thereof

(71) Applicant: The Texas A&M University System, College Station, TX (US)

(72) Inventors: Peng Yu, College Station, TX (US); Chad A. Shaw, Houston, TX (US)

(21) Appl. No.: 15/040,514

(22) Filed: Feb. 10, 2016

Related U.S. Application Data
(60) Provisional application No. 62/114,118, filed on Feb. 10, 2015.

(51) Int. Cl. C12Q 1/68; C12N 15/113

(52) U.S. Cl. CPC C12Q 1/6874; C12N 15/113; C12N 2310/11; C12N 2320/30

(57) ABSTRACT

The present disclosure includes a method for predicting differential alternative splicing events from ribonucleic acid (RNA) sequencing data that includes receiving RNA sequence reads for two or more samples; generating directed acyclic graphs from the RNA sequence reads, wherein each directed acyclic graph represents at least a portion of a gene model; extracting count data from the directed acyclic graphs; and generating differential alternative splicing event information from the count data using a Dirichlet multinomial (DMN) regression.

### CROSS-REFERENCE TO RELATED APPLICATIONS

The present application claims the benefit of U.S. Provisional Patent Application No. 62/114,118, filed on. Feb. 10, 2015, which is incorporated herein by reference.

### STATEMENT OF GOVERNMENTAL SUPPORT

This invention was made with government support awarded by the National Institutes of Health (Grant Numbers U54CA149196, R01CA127857, and F31CA159774). The government has certain rights in the invention.

### FIELD OF THE INVENTION

The present disclosure relates to differential alternative splicing identification. In particular, it relates to a method for modeling, detecting, and identifying differential alternative splicing events.

### BACKGROUND OF THE INVENTION

RNA sequence data may be used to assemble, quantify, and analyze the composition of a gene at a particular moment in time. RNA sequence data may be assembled and reconstructed into a transcript sequence. The RNA sequence data and transcript sequences may be used to estimate the relative gene expression for a particular set of biological conditions. RNA sequence data taken from samples under two or more biological conditions may be used to predict differential expression of particular transcripts between the two conditions.

### SUMMARY OF THE INVENTION

In an embodiment of the invention, a method for predicting differential alternative splicing events from ribonucleic acid (RNA) sequence data includes receiving RNA sequence reads for two or more samples; generating one or more directed acyclic graphs from the RNA sequence reads, wherein each directed acyclic graph represents at least a portion of a gene model; extracting count data from the directed acyclic graphs; and generating differential alternative splicing information from the count data using a Dirichlet multinomial (DMN) regression.

In an embodiment, the invention relates to a method for predicting differential alternative splicing events from ribonucleic acid (RNA) sequence data, comprising: Receiving RNA sequence reads for two or more samples; Generating one or more directed acyclic graphs from the RNA sequence reads, wherein each directed acyclic graph represents at least a portion of a gene model; extracting count data from the directed acyclic graphs; and Generating differential alternative splicing information from the count data using a Dirichlet multinomial (DMN) regression. In an embodiment, the method further comprises generating one or more directed acyclic graphs further comprises: decomposing each directed acyclic graph into alternative splicing types; and summarizing the count data into a count table for each decomposed directed acyclic graph. In an embodiment, the method further comprises aligning the RNA sequence reads to a reference genome or known transcriptome; and quantifying exon and exon-exon junction reads from the RNA sequence reads. In an embodiment, two or more samples are generated under two or more conditions in a multi-factorial experimental design.

In an embodiment, the invention relates to a method for predicting differential alternative splicing events from RNA, comprising: a) sequencing at least one said RNA sample to produce RNA sequence data reads per sample; b) generating one or more directed acyclic graphs from the RNA sequence reads, wherein each directed acyclic graph represents at least a portion of a gene model; c) extracting count data from the directed acyclic graphs; and d) generating differential alternative splicing information from the count data using a Dirichlet multinomial (DMN) regression. In an embodiment, the method further comprises generating one or more directed acyclic graphs further comprises: decomposing each directed acyclic graph into alternative splicing types; and summarizing the count data into a count table for each decomposed directed acyclic graph. In an embodiment, the method further comprises aligning the RNA sequence reads to a reference genome or known transcriptome; and quantifying exon and exon-exon junction reads from the RNA sequence reads. In an embodiment, two or more samples are generated under two or more conditions in a multi-factorial experimental design.

In an embodiment, the invention relates to a method for predicting differential alternative splicing events from ribonucleic acid (RNA) sequence data, comprising: a) sequencing at least one said RNA sample to produce RNA sequence data reads per sample; b) generating one or more directed acyclic graphs from the RNA sequence reads, wherein each directed acyclic graph represents at least a portion of a gene model; c) extracting count data from the directed acyclic graphs; d) generating differential alternative splicing information from the count data using a Dirichlet multinomial (DMN) regression; and e) using said alternative splicing information to create antisense RNA sequence which correspond to said alternative splicing information. In an embodiment, the method further comprises generating one or more directed acyclic graphs further comprises: decomposing each directed acyclic graph into alternative splicing types; and summarizing the count data into a count table for each decomposed directed acyclic graph. In an embodiment, the method further comprises aligning the RNA sequence reads to a reference genome or known transcriptome; and quantifying exon and exon-exon junction reads from the RNA sequence reads. In an embodiment, two or more samples are generated under two or more conditions in a multi-factorial experimental design.

In an embodiment, the invention relates to a method for treating a subject with a disease comprising: a) sequencing at least one said RNA sample from at least one diseased tissue and at least one control sample to produce RNA sequence data reads per sample; b) generating one or more directed acyclic graphs from the RNA sequence reads, wherein each directed acyclic graph represents at least a portion of a gene model; c) extracting count data from the directed acyclic graphs; d) generating differential alternative splicing information from the count data using a Dirichlet multinomial (DMN) regression; e) using said alternative splicing information to create antisense RNA sequences which correspond to said alterative splicing information relevant to said diseased tissue sample; and f) treating said subject with said antisense RNA sequence corresponding to said diseased tissue sample RNA sequence variants so as to alieviate at least one symptom of said disease. In an embodiment, the method further comprises generating one or more directed acyclic graphs further comprises: decomposing each directed acyclic graph into alternative splicing types; and summarizing the count data into a count table for each decomposed directed acyclic graph. In an embodiment, the method further comprises aligning the RNA sequence reads to a reference genome or known transcriptome; and quantifying exon and exon-exon junction reads from the RNA sequence reads. In an embodiment, two or more samples are generated under two or more conditions in a multi-factorial experimental design.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

RNA, for the purpose of the present invention, is considered a single-stranded nucleic acid molecule even where such a molecule may form secondary structures comprising double-stranded RNA portions. Single-stranded RNA molecules may form hybrids together with other RNA molecules, or have in part or over their entire length complementary sequences to form in part or over their entire sequence double-stranded RNA molecules. In particular, RNA encompasses, for the purpose of the present invention, any form of nucleic acid molecule comprised of ribonucleotides, and does not related to a particular sequence or origin of the RNA. Thus RNA can be transcribed in vivo or in vitro by artificial systems, or non-transcribed, spliced or not spliced, processed or not processed, incompletely spliced or processed, independent from its natural origin or derived from artificially designed templates, mRNA, ncRNA, tRNA, rRNA, snRNA, snoRNA, GuideRNA, miRNA, siRNA, piRNA, tasiRNA, tmRNA, macroRNA, macro-ncRNA, obtained by means of synthesis, or any mixture thereof.

As used herein, the term “Ribosome profiling” or “Ribo-Seq” refers to a technique that uses specialized messenger RNA (mRNA) sequencing to determine which mRNAs are being actively translated [1] described by Ingolia (2014) Nat Rev Genet 15(3), 205-213, incorporated herein by reference. It produces a “global snapshot” of all the ribosomes active in a cell at a particular moment, known as a translatome. Consequently, this enables researchers to identify the location of translation start sites, the complement of translated ORFs in a cell or tissue, the distribution of ribosomes on a messenger RNA, and the speed of translating ribosomes [2], described in Weiss (2011) Science 334(6062), 1509-1510, incorporated herein by reference. Ribosome profiling involves similar sequencing library preparation and data analysis to RNA-Seq, but unlike RNA-Seq, which sequences all of the mRNA of a given sequence present in a sample, ribosome profiling targets only mRNA sequences protected by the ribosome during the process of decoding by translation.

The term “disease”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The term “derived from” as used herein, refers to the source of a compound or sequence. In one respect, a compound or sequence may be derived from an organism or particular species. In another respect, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term, “purified” or “isolated”, as used herein, may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways. wherein the compounds interfere with gene expression.

As used herein, the term “shRNA” refers to any small hairpin RNA or short hairpin RNA. Although it is not necessary to understand the mechanism of an invention, it is believed that any sequence of RNA that makes a tight hairpin turn can be used to silence gene expression via RNA interference. Typically, shRNA uses a vector stably introduced into a cell genome and is constitutively expressed by a compatible promoter. The shRNA hairpin structure may also cleaved into siRNA, which may then become bound to the RNA-induced silencing complex (RISC). This complex binds to and cleaves mRNAs which match the siRNA that is bound to it.

As used herein, the term “microRNA”, “miRNA”, or “μRNA” refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed to a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 by or larger are compared.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C0 t or R0 t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tm” is used in reference to the “melting temperature.” The melting temperature is the temperature at which a population of double-stranded nucleic acid molecules becomes half dissociated into single strands. As indicated by standard references, a simple estimate of the Tm value may be calculated by the equation: Tm=81.5+0.41 (% G+C), when a nucleic acid is in aqueous solution at 1M NaCl. Anderson et al., “Quantitative Filter Hybridization” In: Nucleic Acid Hybridization (1985) [3]. More sophisticated computations take structural, as well as sequence characteristics, into account for the calculation of Tm.

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of a target sequence of interest. In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Amplification” is defined as the production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y. [4].

As used herein, the term “polymerase chain reaction” (“PCR”) refers to the method of K. B. Mullis U.S. Pat. Nos. 4,683,195 [5] and 4,683,202 [6], herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers; to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

The term “in operable combination” as used herein, refers to any linkage of nucleic acid sequences in such a manner that a nucleic acid molecule capable of directing the transcription of a given gene and/or the synthesis of a desired protein molecule is produced. Regulatory sequences may be operably combined to an open reading frame including but not limited to initiation signals such as start (i.e., ATG) and stop codons, promoters which may be constitutive (i.e., continuously active) or inducible, as well as enhancers to increase the efficiency of expression, and transcription termination signals.

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987) [7]. Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site. Sambrook, J. et al., In: Molecular Cloning: A Laboratory Manual, 2nd ed., Cold Spring Harbor laboratory Press, New York (1989) pp. 16.7-16.8. A commonly used splice donor and acceptor site is the splice junction from the 16S RNA of SV40 [8].

The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists. J. Sambrook et al. (1989) In: Molecular Cloning: A Laboratory Manual, Cold Spring Harbor Press, N.Y., pp 9.31-9.58 [8].

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligodeoxyribonucleotide probe or DNA probe to detect RNA species complementary to the probe used. Northern blots are a standard tool of molecular biologists. J. Sambrook, J. et al. (1989) supra, pp 7.39-7.52 [8].

The term “reverse Northern blot” as used herein refers to the analysis of DNA by electrophoresis of DNA on agarose gels to fractionate the DNA on the basis of size followed by transfer of the fractionated DNA from the gel to a solid support, such as nitrocellulose or a nylon membrane. The immobilized DNA is then probed with a labeled oligoribonuclotide probe or RNA probe to detect DNA species complementary to the ribo probe used.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation. The term “suspected of having”, as used herein, refers a medical condition or set of medical conditions (e.g., preliminary symptoms) exhibited by a patient that is insufficent to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subj ect.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatiblity is evaluated according to the application for which it was designed: for example; a bandage is regarded a biocompable with the skin, whereas an implanted medical device is regarded as biocompatible with the internal tissues of the body. Preferably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “bioerodible” as used herein, refers to any material that is mechanically worn away from a surface to which it is attached without generating any long term inflammatory effects such that the molecular structure has not been altered. In one sense, bioerosin represents the final stages of “biodegradation” wherein stable low molecular weight products undergo a final dissolution.

The term “bioresorbable” as used herein, refers to any material that is assimilated into or across bodily tissues. The bioresorption process may utilize both biodegradation and/or bioerosin.

The term “biostable” as used herein, refers to any material that remains within a physiological environment for an intended duration resulting in a medically beneficial effect.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings included in the present application are incorporated into, and form part of, the specification. They illustrate embodiments of the present invention and, along with the description, serve to explain the principles of the invention. The drawings are only illustrative of embodiments of the invention and do not limit the invention.

FIG. 1 is a diagram of a method for predicting differential alternative splicing events from RNA sequence data, according to embodiments of the disclosure.

FIG. 2A is an exemplary diagram of a gene segment having eight exons shown, according to embodiments of the disclosure.

FIG. 2B displays an exemplary directed acyclic graph of a gene model, according to embodiments of the disclosure.

FIG. 2C displays an exemplary directed acyclic graph that has been simplified from FIG. 2B, according to embodiments of the disclosure.

FIG. 2D displays two exemplary graphs showing a skipping event and a mutually exclusive exon junction, according to embodiments of the disclosure.

FIG. 3A is a directed acyclic graph with reads for a condition 1, according to embodiments of the disclosure.

FIG. 3B is a directed acyclic graph with reads for a condition 2, according to embodiments of the disclosure.

FIG. 3C is a count table for the count data of FIG. 3A and FIG. 3B, according to embodiments of the disclosure.

FIG. 4 shows a workflow of differential alternative splicing analysis using DASplice. DASplice takes single-end or paired-end RNA-seq datasets with biological replicates for analysis. An exon skipping event is shown. Yellow indicates the alternative spliced exon; blue and red indicate the flanking constitutive exons. The exons are number as E1, E2 and E3. RNA-seq reads are aligned to the reference genome. Splice graphs can be constructed using the splice reads and the gene annotations. Multiple isoforms are inferred from a splice graph with alternative splicing. A count table is compiled for each alternative splicing event by counting the number of sequence fragments unambiguously supporting all isoforms. A likelihood ratio test based on the Dirichlet multinomial distribution is used to test for the differential usage of isoforms between treatments or study groups, resulting in p-values for each alternative splicing event. False Discovery Rate q-values are determined to account for multiple hypothesis testing. Based on the count table, the log-odds-ratio between the usage ratios of the two isoforms of the two genotypes can be computed (not shown in the figure), which can be used to access the effect sizes of the alternative splicing events.

FIG. 5 shows a simulation study to compare DASplice and Fisher's exact test. DASplice significantly outperforms Fisher's exact test method based on the AUC of the ROC curve (i.e. the sensitivity versus (1-specificity) plot).

FIGS. 6A & 6B show a confirmation of a DASplice identified event in Postn (E16-E18). FIG. 6A shows a visualization of the junction RNA-Seq reads in bigWig format in the UCSC genome browser for Postn. FIG. 6B shows a RT-PCR confirmation result for Postn. Isoform depiction colors indicate the constitutive exons in blue and the variable exon in yellow. Gel images display the raw pixel net intensity values. (“*”: p<0.05).

FIGS. 7A & 7B shows a confirmation of a DASplice identified event in Dst (E19-E21). FIG. 7A shows a visualization of the junction RNA-Seq reads in bigWig format in the UCSC genome browser for Dst. FIG. 7B shows a RT-PCR confirmation result for Dst. Isoform depiction colors indicate the constitutive exons in blue and the variable exon in yellow. Gel images display the raw pixel net intensity values.

FIGS. 8A & 8B show a confirmation of a DASplice identified event in Myh11 (E42-E44). FIG. 8A shows a visualization of the junction RNA-Seq reads in bigWig format in the UCSC genome browser for Myh11. FIG. 8 shows a RT-PCR confirmation result for Myh11. Isoform depiction colors indicate the constitutive exons in blue and the variable exon in yellow. Gel images display the raw pixel net intensity values.

FIG. 9 shows examples of common alternative splicing events. Note that alternative promoters are regulated by transcriptional mechanisms. Since they can be studied using RNA-seq data, DASplice is able to consider them, and this is an important advantage of the method compared to alternatives.

FIG. 10A-10D show Graph notation of alternative splicing events, with 5′ end on the left and 3′ end on the right. FIG. 10A shows exon skipping; FIG. 10B shows alternative 5′ splice sites; FIG. 10C shows alternative promoters. FIG. 10D shows for the 5′ end.

FIG. 11A-11D show that graph representation can interpret complex splicing types. FIG. 11A shows a tandem exon skipping type; FIG. 11B shows a mutually exclusive exon type, FIG. 11C shows an intron retention type; FIG. 11D shows a coupled alternative 5′ splice sites and alternative 3′ splice sites type.

FIG. 12A-12E shows the detection of alternative splicing types from a gene model using a graphical algorithm. FIG. 12A shows An illustration of a gene model with a skipped exon and two mutually exclusive exons. FIG. 12B shows DAG gene model representation. FIG. 12C shows Splice junctions (edges) not involved with alternative splicing are collapsed to simplify subsequent graph manipulation. FIG. 12D shows the collapsed graph is cut at certain nodes so that the resulted components correspond to alternative splicing types. FIG. 12E shows the alternative splicing types are determined from these components.

FIG. 13 shows the number of RNA-seq reads on a given isoform can be counted, forming the count table that will be used for statistical testing. DASplice can handle multiple replications and complex alternative splicing types.

FIG. 14A & 14B show confirmation of a DASplice identified event in Dst (E46-E52). FIG. 14A shows UCSC genome browser tracks from Green and Red replicates for Dst displaying differential alternative splicing of multiple cassette exons. FIG. 14B show a PCR assay of the two possible alternative isoforms generated from multiple cassette exon inclusion/exclusion and percent of exon E51 inclusion calculation.

FIGS. 15A & 15B shows confirmation of a DASplice identified event in Dcn (E1-E3). FIG. 15A shows UCSC genome browser tracks from Green and Red replicates for Dcn displaying differential alternative splicing at exons one and two. FIG. 15B shows PCR assay of the two possible alternative isoforms generated from different promoter usage and percent of exon E2 utilization calculation.

FIGS. 16A & 16B show confirmation of a DASplice identified event in Cttn (E10-E12). FIG. 16A shows UCSC genome browser tracks from Green and Red replicates for Cttn displaying differential cassette exon usage at exon 11. FIG. 16B shows PCR assay of the two possible alternative isoforms generated from cassette exon inclusion/exclusion and percent of exon E11 inclusion calculation.

FIGS. 17A & 17B show confirmation of a DASplice identified event in Mbtps2 (E1-E3). FIG. 17A shows UCSC genome browser tracks from Green and Red replicates for Mbtps2 displaying differential cassette exon usage at exon two. FIG. 17B show PCR assay of the two possible alternative isoforms generated from cassette exon inclusion/exclusion and percent of exon E2 inclusion calculation.

FIGS. 18A & 18B show confirmation of a DASplice identified event in Zdhhc5 (E9-E11). FIG. 18A shows UCSC genome browser tracks from Green and Red replicates for Zdhhc5 displaying differential cassette exon usage at exon ten. FIG. 18B shows PCR assay of the two possible alternative isoforms generated from cassette exon inclusion/exclusion and percent of exon E10 inclusion calculation.

FIGS. 19A & 19B shows a Confirmation of a DASplice identified event in Usp48 (E15-E17). FIG. 19A shows UCSC genome browser tracks from Green and Red replicates for Usp48 displaying alternative poly adenylation utilization at E16 coupled to 3′ splice site choice. FIG. 19B shows PCR assay of the two possible isoforms generated from differential three prime usage and percent exon 17 utilization calculation.

FIG. 20 shows UCSC genome browser track of the DEXSeq prediction Dlc1. Green sample tracks are colored in green and Red sample tracks are colored in red.

FIG. 21 shows UCSC genome browser track of the DEXSeq prediction Hc. Green sample tracks are colored in green and Red sample tracks are colored in red.

FIG. 22 shows UCSC genome browser track of the DEXSeq prediction Abi3bp. Green sample tracks are colored in green and Red sample tracks are colored in red.

FIG. 23 shows UCSC genome browser track of the DEXSeq prediction Sprx. Green sample tracks are colored in green and Red sample tracks are colored in red.

FIG. 24 shows UCSC genome browser track of the DEXSeq prediction Obfc1. Green sample tracks are colored in green and Red sample tracks are colored in red.

FIG. 25 shows UCSC genome browser track of the DEXSeq prediction Pdgfra. Green sample tracks are colored in green and Red sample tracks are colored in red.

FIGS. 26A & 26B show confirmation of a DEXSeq identified event. FIG. 26A shows UCSC genome browser tracks from Green and Red replicates for Mmp19 displaying differential alternative splicing at intron 11. FIG. 26B shows PCR assay of the two possible alternative isoforms generated from differential splicing resulting in inclusion/exclusion of exon E11.

FIGS. 27A & 27B shows confirmation of a MATS identified event in Pkp4. FIG. 27A shows UCSC genome browser tracks of exons E27, E28 and E29. MATS indicates that there is an decreased usage of E28 in the Green replicates than in the Red replicates. FIG. 27B shows the PCR experiment confirmed that there was no statistical significant usage change in this alternative splicing event between the Green replicates and Red replicates.

FIGS. 28A & 28B shows confirmation of a MATS identified event 5530601H04Rik (E7-E9,E13). FIG. 28A shows UCSC genome browser tracks from Green and Red replicates for 5530601H04Rik displaying differential alternative splicing at mutually exclusive exons eight and nine. FIG. 28B shows PCR assay of the four alternative isoforms generated from differential splicing resulting in inclusion/exclusion of exons eight and nine.

FIGS. 29A & 29B shows confirmation of a MATS identified event Prosc (E2,E4,E6,E9). FIG. 29A shows UCSC genome browser tracks from Green and Red replicates for Prosc displaying differential alternative splicing at mutually exclusive exons E4 and E6. FIG. 29B shows PCR assay of the four alternative isoforms generated from differential splicing resulting in inclusion of exons four and six.

FIGS. 30A & 30B shows confirmation of a MATS identified event Chd2 (E12-E15). FIG. 30A shows UCSC genome browser tracks from Green and Red replicates for Chd2 displaying differential alternative splicing at mutually exclusive exons E13 and E14. FIG. 30B show PCR assay of the four alternative isoforms generated from differential splicing resulting in inclusion/exclusion of exons E13 and E14.

FIGS. 31A & 31B shows confirmation of a MATS identified event Prosc (E2-E4). FIG. 31A shows UCSC genome browser tracks from Green and Red replicates for Prosc displaying differential cassette exon usage at exon three. FIG. 31B show PCR assay of the two possible alternative isoforms generated from cassette exon inclusion/exclusion and percent of exon E3 inclusion calculation. The direction of change indicated by the PCR assay is opposite to the prediction made by MATS.

### DETAILED DESCRIPTON OF THE INVENTION

According to embodiments of the disclosure, a method for predicting differential alternative splicing events from ribonucleic acid (RNA) sequence data may involve representing the data using one or more directed acyclic graph (DAG) data structures, modeling the data using a Dirichlet multinomial (DMN) distribution, and analyzing the data using a DMN regression method. RNA sequence count data may be extracted from a DAG representing a gene model. The DAG may be generated from raw data, as well as existing gene annotation. This DAG representation may represent complex alternative splicing types and allow for development of an improved set of hypotheses for the discovery of differential alternative splicing events. The Dirichlet multinomial distribution model may preserve important RNA sequence data by accounting for over-dispersion of the data. Using the DMN regression, complex design information may be represented in matrix form, enabling analysis for multi-factorial experimental designs. By utilizing this DAG representation and DMN distribution/regression method, RNA sequence data may be used to more accurately predict differential alternative splicing events.

According to embodiments of the disclosure, a method for predicting differential alternative splicing events from ribonucleic acid (RNA) sequence data may involve representing the data using one or more directed acyclic graph (DAG) data structures, modeling the data using a Dirichlet multinomial (DMN) distribution, and analyzing the data using a DMN regression method. RNA sequence count data may be extracted from a DAG representing a gene model. The DAG may be generated from raw data, as well as existing gene annotation. This DAG representation may represent complex alternative splicing types and allow for development of an improved set of hypotheses for the discovery of differential alternative splicing events. The Dirichlet multinomial distribution model may preserve important RNA sequence data by accounting for over-dispersion of the data. Using the DMN regression, complex design information may be represented in matrix form, enabling analysis for multi-factorial experimental designs. By utilizing this DAG representation and DMN distribution/regression method, RNA sequence data may be used to more accurately predict differential alternative splicing events. In some embodiments, the invention comprises initial sequencing of RNA sequences before analysis. Some of the challenges in the field of RNA sequences are described by Ozsolak, 2011, Nat. Rev. Genet. 12(2), 87-98 [9], incorporated herein by reference. In one embodiment, RNA sequencing may comprise the use of RNA-Seq—quantitative measurement of expression through massively parallel RNA-sequencing, as described in Wilhelm, 2009, Methods 48(3), 249-257 [10] and described by De Wit et al. (2012) Mol. Ecol. Resour. 12(6), 1058-1067 [11], incorporated herein by reference. In one embodiment, the invention relates to a method may be integrated into any technology that can generate sequence reads. In one embodiment, the method may be used in not only RNA-seq data (which come from two protocols poly-A selection and rRNA depletion). In one embodiment, the method may also be used for Ribosome profiling data, to identify differentially alternatively spliced isoforms that are being translated. In one embodiment, said RNA sequences may be identified by deep sequencing of ribosome-protected mRNA fragments as described in Ingolia et al. (2012) Nat Protoc 7(8), 1534-1550 [12], incorporated herein by reference. In one embodiment, said method may be combined with optogenetics to identify candidate genes that affect neural circuit activity, such as been described in Stuber 2013 Pharmacol Rev 65(1), 156-170 [13], herein incorporated by reference. In one embodiment, the invention comprises sequencing of DNA and subsequent translation of said DNA into RNA sequences for analysis.

In one embodiment, the RNA analysis method described herein may be used to identify novel splicing isoforms related to disease states. In one embodiment, said identified isoforms may be used to produe interfering RNA to treat said disease states. In one embodiment, said disease states include cancers, such as breast cancer and skin cancer as RNA sequencing of cancer has been shown to reveal novel splicing alterations as described in Eswaran (2013) Sci Rep 3, 1689 [14], incorporated herein by reference. In one embodiment, said method may identify specific isoforms may be knock down by RNAi to suppress cancer growth. The knockdown of Akt isoforms by RNA silencing suppresses the growth of human prostate cancer cells in vitro and in vivo is described by Sasaki 2010 [15], incorporated herein by reference. In one embodiment, said method may be used to identify splicing isoforms that may be regulated by certain treatments in plant, for example stress condition, etc. For example, Global insights into high temperature and drought stress regulated genes by RNA-Seq in economically important oilseed crop Brassica juncea as described by Bhardwaj 2015 [16], incorporated herein by reference. In one embodiment, said method may be used to identify splicing changes that may be affected by mouse mutations, such as those described in Cheng 2014 [17].

Small interfering RNAs (siRNAs), which downregulate gene expression guided by sequence complementarity, can be used therapeutically to block the synthesis of disease-causing proteins. For example Wittrup et al., (2015) Nat. Rev. Genet. 16(9), 543-552 describes some current examples of siRNA therapies [18], incorporated herein by reference.

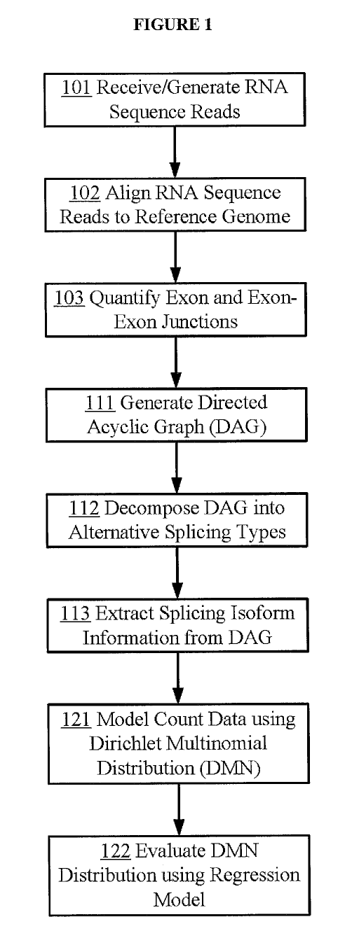

The short RNA sequence reads may be aligned to a reference genome or known transcriptome, as in 102 in FIG. 1, and filtered for uniquely-mapped reads. The reference genome may be taken from a RNA sequence library or reference transcript database. The sequence data may be aligned using unspliced read alignment methods, such as Burrows-Wheeler transform, spliced read alignment methods, such as seed-and-extend or exon-first, or gene annotations.

The short RNA sequence reads may be aligned to a reference genome or known transcriptome, as in 102 in FIG. 1, and filtered for uniquely-mapped reads. The reference genome may be taken from a RNA sequence library or reference transcript database. The sequence data may be aligned using unspliced read alignment methods, such as Burrows-Wheeler transform, spliced read alignment methods, such as seed-and-extend or exon-first, or gene annotations.

The aligned exon and exon-exon junction reads may be quantified, as in 103 in FIG. 1. This quantification may be done by counting the reads to determine the relative abundance of exon and exon-exon junction reads in each sample. The base/read counts of exon and exon-exon junctions may be computed using a gene annotation and normalized.

A directed acyclic graph (DAG) may be generated for the RNA sequence data, as in 111 in FIG. 1. A directed acyclic graph is a partially-ordered graph with no directed cycles. A DAG may be uniquely appropriate for representing gene transcription due to the ordering of exons by chromosomal coordinates and known transcription direction of a gene (directed acyclic; partially-ordered). A DAG consists of nodes, each representing an isoform, such as an exon. The nodes are connected by edges, each representing a splicing event, such as an exon junction. The DAG may be generated from a gene model or from raw data. The RNA sequence reads may be uniquely mapped to the alternative splicing types and isoforms represented in the DAG.

Each directed acyclic graph may be simplified and decomposed into alternative splicing types, as in 112 in FIG. 1. The DAG for a gene model may be segregated into weakly biconnected components. Each of the weakly biconnected components may represent an alternative splicing type.

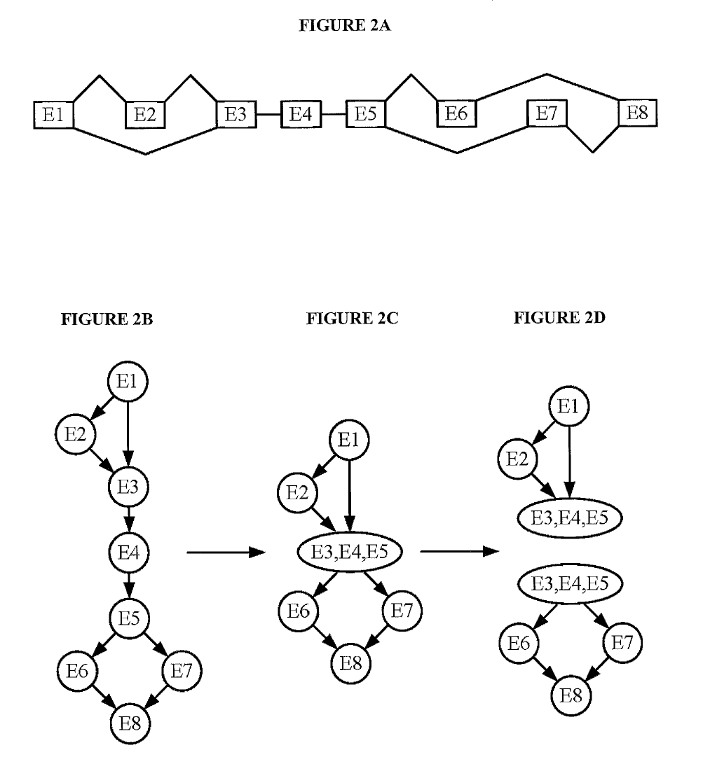

FIG. 2A, FIG. 2B, FIG. 2C, and FIG. 2D are exemplary diagrams of the directed acyclic graph construction and representation of 111 and 112 in FIG. 1 and described above, according to embodiments of the disclosure. FIG. 2A is an exemplary diagram of a gene segment having eight exons shown. The gene has a skipped exon E2 and two mutually exclusive exons E6 and E7. FIG. 2B, FIG. 2C, and FIG. 2D are three exemplary graphs showing construction, simplification, and decomposition of the gene segment of FIG. 2A. FIG. 2B displays an exemplary directed acyclic graph of a gene model. The gene model of FIG. 2A may be converted into the directed acyclic graph of FIG. 2B. Because E3-E4-E5 does not contain any alternative splicing paths, the DAG may be simplified to FIG. 2C. The node of E3-E4-E5 may be split into both a root and a leaf, decomposing to two weakly biconnected components as shown in FIG. 2D. These components may be classified into alternative splicing events; in this example, the top graph may be characterized as an exon skipping event and the bottom graph may be characterized as mutually exclusive exons.

Returning to FIG. 1, splicing isoform information may be extracted from the directed acyclic graphs, as in 113. A DAG for an alternative splicing event may contain RNA sequence reads from which splice type information may be extracted. This extraction may be used to summarize uniquely mapped RNA sequence reads into count data in the form of count tables for each alternative splicing type.

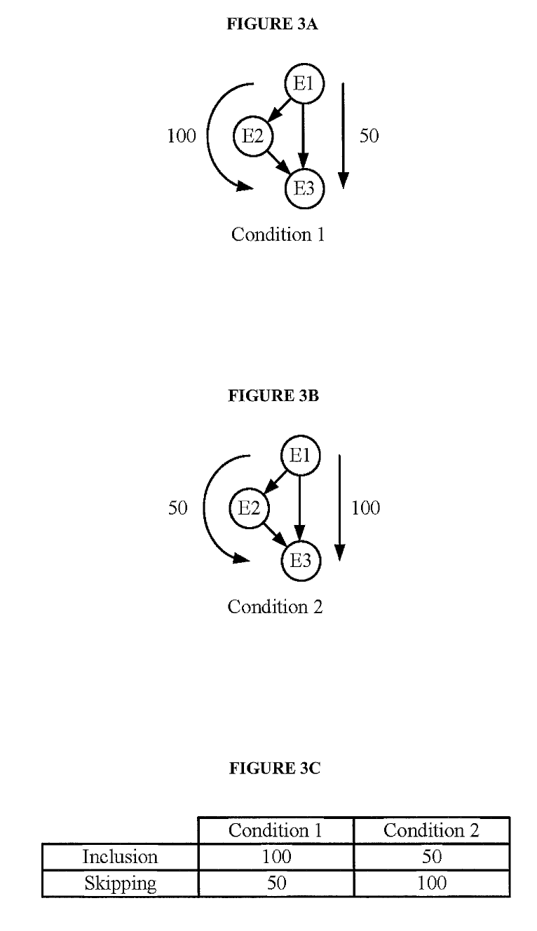

FIG. 3A and FIG. 3B are two exemplary directed acyclic graphs of skipping events, according to embodiments of the disclosure. FIG. 3A is a DAG for a condition 1, where 50 reads are mapped for E1-E3 (skipping) and 100 reads are mapped for E1-E2-E3 (inclusion). FIG. 3B is a DAG for a condition 2, where 100 reads are mapped for E1-E3 and 50 reads are mapped for E1-E2-E3. FIG. 3C is a count table for the mapped reads of FIG. 3A and FIG. 3B, according to embodiments of the disclosure. In this example, E2 is four times as likely to be skipped under condition 2 as condition 1.

Returning to FIG. 3, the count data is modeled and evaluated using a Dirichlet multinomial distribution, as in 121 in FIG. 1. The Dirichlet distribution is a count data distribution that models how a set number of objects are allocated in a specified number of categories. The Dirichlet distribution may be appropriate for modeling count data generated from RNA sequence reads, for which the actual variance tends to be greater than the nominal variance of a multinomial distribution. The Dirichlet multinomial (DMN) model extends the multinomial distribution to model overdispersion by using a Dirichlet prior. The probability mass function of the DMN distribution may be expressed as follows:

$${f}_{DMN} x ; N , α = \frac{N !}{{\sum }_{k = 1}^{K} {x}_{k} !} \frac{Γ ( {\sum }_{i = 1}^{K} {α}_{i} )}{Γ ( {\sum }_{i = 1}^{K} {α}_{i} + N )} {\sum }_{k = 1}^{K} \frac{Γ ( {α}_{k} + {x}_{k} )}{Γ ( {α}_{k} )}$$

where K represents the number of categories, x represents an observation of a particular category in a trial, xₖ represents the number of observations of a particular category, N represents the total number of independent trials, and α represents the Dirichlet prior. The DMN model includes proportion parameters representing the probability of a trial belonging to a particular splicing event and a dispersion parameter representing the variance of the distribution, including overdispersion. This parameterization may allow for smooth transition between an overdispersed case and a non-overdispersed case. The log likelihood function may be written as follows:

$$ln Lp , ψ ; x = - ( ln Γ1ψ + N - ln Γ1ψ ) + {\sum }_{k = 1}^{K} ( ln Γ 1 \frac{ψ}{{p}_{k}} + {x}_{k} - ln Γ1 \frac{ψ}{{p}_{k}} )$$

where ψ represents the dispersion parameter for the distribution and pₖ represents the proportion parameter for a particular category.

The log gamma differences in the log-likelihood function may be approximated based on a truncated series. For small dispersion parameters, the log gamma difference for the dispersion parameter terms may be small compared to the log gamma terms themselves, which may create truncation errors when floating point arithmetic is used. The log gamma differences for the dispersion and proportion parameters may be approximated by asymptotically expanding the paired log gamma functions and reducing the log gamma difference to a truncated series having Bernoulli polynomials. This approximation may reduce the error caused by floating point arithmetic and enable a smoother transition between the overdispersed (large dispersion parameter) and non-overdispersed (small dispersion parameter) cases. Each paired log gamma difference may be approximated as follows:

$$ln Γ ( 1 a + b ) - ln Γ ( 1 a ) \approx - b ln a + {\sum }_{n = 2}^{m} \frac{{( - 1 )}^{n} {φ}_{n} ( b )}{n ( n - 1 )} {a}^{n} - 1$$

where a may represent ψ or ψ/pₖ, b may represent N or xₖ, φₙ(·) may represent a Bernoulli polynomial of the nth degree, and m may represent a parameter that controls the error bound. The error may be bounded by:

$${\sum }_{n = m + 1}^{∞} \frac{1}{n - 1} {δ}^{n}$$

where δ is a constant less than 1. The error bound may be simplified to xy≦δ for the computation of the DMN log likelihood function.

The Dirichlet multinomial distribution may be evaluated using a regression analysis, as in 122. The log gamma approximation may be analytically continued into the whole parameter domain of the DMN log-likelihood function by incrementally evaluating the DMN log-likelihood function on a mesh. The level of mesh may be selected so that the log-likelihood function may meet the error bound condition. The mesh log-likelihood function of an observation may be represented as follows:

$$ln L ( p , ψ ; x ) = {\sum }_{l = 1}^{L} ln L ( {p}^{l - 1} , {ψ}^{l - 1} ; {x}^{( l )} )$$

where L represents the nonzero observations meeting the error bound.

The total log-likelihood function for all observations may be expressed as the summation of the log-likelihood function of each observation. The observations (N×L), proportion parameters (N×L), and dispersion parameters (N×1) may be represented in matrices, according to the following equations:

$$ln L ( p , ψ ; x ) = {\sum }_{l = 1}^{L} ln L ( {p}_{i} , {ψ}_{i} ; {x}_{i} )$$

$${x}_{N \times L} = \begin{matrix}{x}_{1} \\ ⋮ \\ {x}_{N}\end{matrix} , \\ {p}_{N \times 1} = \begin{matrix}{p}_{1} \\ ⋮ \\ {p}_{N}\end{matrix} , \\ {ψ}_{N \times L} = \begin{matrix}{ψ}_{1} \\ ⋮ \\ {ψ}_{N}\end{matrix}$$

The DMN regression may be based around a framework similar to a generalized linear model (GLM) regression involving the maximum likelihood of regression parameters in the log-likelihood function. An appropriate link function may be selected for each of the parameters based on the baseline-category logits model for multinomial distributions. A likelihood ratio test may be used to determine any significant changes of the proportion parameters between two or more groups of samples generated under different biological conditions. Samples generated from multiple conditions may be used for multi-factorial designs. The DMN regression may detect a set of differential alternative splicing events, which have event types derived in the DAG generation step above, with p-values calculated indicating statistical significance, and may include log-odds-ratio information for isoform usage and corresponding proportion values.

The method described above may be applied to multi-factorial design experiments to test for the interaction effect among groups of samples, among other tests. For example, comparative analysis of groups of samples may remove the effect of extraneous variables, which may increase the power of the analysis.

Aspects of the invention may be implemented by computer systems informed by human selection and input. For example, the DMN regression framework may be coded into software instructions and executed by a computer. Certain parameters that the method uses for predicting alternative differential splicing events, such as error bound parameters and categories, may be selected by a user operating the software.

Although the present invention has been described in terms of specific embodiments, it is anticipated that alterations and modifications thereof will become apparent to those skilled in the art. Therefore, it is intended that the following claims be interpreted as covering all such alterations and modifications as fall within the true spirit and scope of the invention.

As will be appreciated by one skilled in the art, aspects of the present disclosure may be embodied as a system, method, or computer program product. Accordingly, aspects of the present disclosure may take the form of an entirely software embodiment (including firmware, resident software, micro-code, etc.) or an embodiment combining software and hardware aspects that may all generally be referred to herein as a “circuit,” “module,” “device,” or “system.” Furthermore, aspects of the present inventions may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium including, but not limited to, wireless, wire line, optical fiber cable, RF, etc., or any suitable combination of the foregoing.

Computer program code for carrying out operations for aspects of the present disclosure may be written in any combination of one or more programming languages, including an object-oriented language such as Java, C++, Python, or the like, and conventional procedural programming languages, such as “C” and “R” programming languages or similar programming languages. The program code may execute entirely on the user's computer, partly on the user's computer, as a standalone software package, partly on the user's computer and partly on a remote computer or server, or entirely on the remote computer or server. In the last scenario, the remote computer may be connected to the user's computer through any type of network, including a local area network (LAN), a wide area network (WAN), a cellular network, or the connection may be made to an external computer (for example, through the internet using an Internet Service Provider).

Aspects of the present disclosure have been described above with reference to flowchart illustrations, equations, and/or block diagrams of methods, apparatus (systems), and computer program products, according to embodiments of the disclosure. It will be understood that blocks of the flowchart illustrations and/or block diagrams, and the equations, can be implemented partly or wholly by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart, block diagrams, and/or equations.

These computer program instructions may also be stored in a computer readable medium that can direct a computer, other programmable data processing apparatus, or other device to function in a particular manner, such that the instructions stored in the computer readable medium produce an article of manufacture including instructions which implement the functions/acts/operations specified in the flowchart, block diagram, and/or equation.

The computer program instructions may also be loaded onto a computer, other programmable data processing apparatus, or other device to cause a series of operational steps to be performed on the computer, other programmable apparatus, or other device to produce a computer implemented process such that the instructions which execute on the computer or other programmable apparatus provide processes for implementing the functions/acts/operations specified in the flowchart, block diagram, or equation.

The flowchart and block diagram in FIG. 1 illustrates the architecture, functionality, and operation of possible implementations of systems, methods, and computer program products according to various embodiments of the present disclosure. In this regard, each block in the flowchart and block diagram may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently or the blocks may sometimes be executed in the reverse order, depending upon the functionality involved. It will also be noted that each block of the block diagrams and/or flowchart illustrations, can be implemented by special purpose hardware-based systems that perform the specified functions, acts, or operations, or combinations of special purpose hardware and computer instructions.

### EXAMPLES

The following examples are provided in order to demonstrate and further illustrate certain preferred embodiments and aspects of the present invention and are not to be construed as limiting the scope thereof.

### Ribosome Profiling

Procedure:
1. Lyse the cells or tissue and isolate the mRNA molecules bound to ribosomes.
2. Immobilize complexes. This is commonly performed with cycloheximide but other chemicals can be employed. It is also possible to forgo translation inhibitors with translation-incompetent lysis conditions.
3. Using ribonucleases, digest the RNA not protected by ribosomes.
4. Isolate the mRNA-ribosome complexes using sucrose gradient density centrifugation or specialized chromatography columns.
5. Phenol/chloroform purification of mixture to remove proteins.
6. Size-select for previously-protected mRNA fragments.
7. Ligate 3′ adapter to fragments.
8. Subtract known rRNA contaminants (optional).
9. Reverse transcribe RNA to cDNA using reverse transcriptase.
10. Amplify in strand-specific manner.
11. Sequence reads.
12. Align sequence results to genomic sequence to determine translational profile, for example see Ingolia et al. 2009 [19].
13. Apply RNA sequences to the analysis described herein, such as applying the DASplice process.

### DASplice Example

High-throughput RNA sequencing (RNA-Seq) represents a powerful platform for discovering differential alternative splicing. However, robust detection of alternative splicing in the context of biological variation and complex study designs remains a challenge. To address this challenge, a new method was developed called DASplice based on a directed acyclic graph data representation together with a novel count data statistical analysis method. Simulations and wet bench experiments demonstrate the method's advantages. DASplice can discover both simple and complex alternative splicing events; it is also highly sensitive to detect subtle changes that are common in alternative splicing, and the method is ideal for data sets exhibiting large within-treatment variation. DASplice paves the way for comprehensive genome-wide discovery of differential alternative splicing in various tissues and cell types as well as multifactorial studies.

### Introduction

Alternative splicing is a mechanism that allows one gene to encode multiple proteins, thus increasing the diversity of transcriptomes and proteomes arising from a single genome [20]. Alternative splicing is frequently observed in higher eukaryotes, though its complexity was not fully appreciated until the advent of sequencing technology such as RNA-Seq that permits direct observation of RNA expression and splicing. Recent studies applying RNA-Seq in human samples showed that 90% of human genes undergo alternative splicing [21, 22]. Disruption of alternative splicing causes human diseases such as myotonic dystrophy type 1 (a muscle disorder where a group of alternative splicing events are altered causing abnormalities in skeletal muscle and heart development) [23, 24], retinitis pigmentosa (a retinal disease harboring mutations in known pre-mRNA splicing factors) [25] and cancer [26].

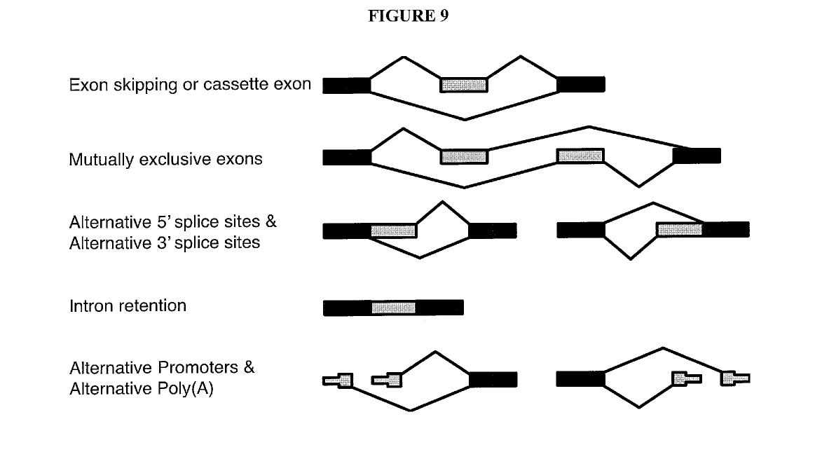

A major difficulty in the analysis of RNA-Seq data stems from the intrinsic complexity of alternative splicing events. The most commonly studied alternative splicing event is exon skipping, where the mature mRNA transcript may have an exon spliced in or out (top example in FIG. 9). In addition to exon skipping, there are many additional alternative splicing events (FIG. 9). In fact, the number of splicing events can be many orders of magnitude larger than the number of genes [27]. A systematic method is necessary to capture this complexity and achieve a comprehensive analysis of alternative splicing.

Although a number of tools for analyzing alternative splicing have been developed, these tools have various deficiencies significantly limiting their utility in biomedical research. Mixture of Isoforms (MISO) [28] is able to estimate isoform expression levels within a sample based on a statistical model, but it is unable to account for variability across experimental replicates. Shen et al. proposed a Bayesian framework called Multivariate Analysis of Transcript Splicing (MATS) for detecting differential alternative splicing from RNA-Seq data [29]. Because MATS uses a Bayesian framework, MATS inherits the limitations of Bayesian statistics [30], such as the difficulty in specifying an appropriate prior and the long runtime of Markov chain Monte Carlo (MCMC) simulations. The DEXSeq [31] method considers the count nature of RNA-Seq data and biological replicates, but takes an exon-centric approach and does not exploit information available at splice junctions. As a consequence, DEXSeq is able to detect large changes in alternative splicing, but may miss many smaller changes that are commonly observed in alternative splicing. Moreover, DEXSeq fails to provide information on splicing event types, which makes it difficult to design molecular assays to confirm detected events. This lack of clear mapping between statistical evidence of alternative splicing and a specific splicing hypothesis that can be experimentally tested is an important consideration. Another popular RNA-Seq data analysis method is Cufflinks [32]. However, this method is directed toward differential transcript expression analysis not specifically tailored to analyze differential alternative splicing. It estimates transcript expression levels as an intermediate step, but the short length of RNA-Seq reads makes it difficult to provide accurate isoform estimates, thereby rendering measurements imprecise [33].

To address the challenge of alternative splicing analysis, we developed a novel method called DASplice. This method combines a directed acyclic graph (DAG) representation of RNA-Seq data as well as a new method for discrete count data statistical modeling. In DASplice, RNA-Seq reads or fragment count data are recorded on a DAG, allowing gene models to be derived from raw data and not restricted to existing, and often incomplete, gene annotations. The DAG-based representation is ideal for RNA-Seq analysis of alternative splicing analysis because it is flexible and able to represent complex alternative splicing types. DASplice also uses a rigorous statistical inference methodology enabled by a recent development in count data statistical modeling [34] that preserves critical information in the RNA-Seq discrete observations. Here, the usefulness of DASplice is demonstrated on an RNA-Seq data set derived from an in vivo mouse model displaying paracrine hedgehog (Hh) signaling between epithelial cells in the mammary gland, leading to increased proliferation in the responding cell population. The effectiveness of DASplice and its ability to detect alternative splicing changes missed by other alternative splicing analysis methods has been confirmed.

Herein the effectiveness of DASplice and its ability to detect alternative splicing changes missed by other alternative splicing analysis methods is demonstrated. The biological data set to which the method is applied is a RNA-Seq data derived from an in vivo mouse model expressing a constitutively activated form of the Smoothened (SMO) protein, which serves as the main signal transducer of the Hedgehog (Hh) signaling network in the presence of the Hh morphogen in the mammary gland. In breast cancers, SMO is ectopically expressed in ˜70% of ductal carcinoma in situ (DCIS) and ˜30% of invasive breast cancers (IBC) [35]. In canonical Hh network signaling, the Smoothened (SMO) oncoprotein serves as the main signal transducer of this signaling pathway in the presence of the Hh morphogen. In breast cancers, SMO is ectopically expressed in ˜70% of ductal carcinoma in situ (DCIS), and ˜30% of invasive breast cancers (IBC) [35]. Overexpression of a Cre-recombinase-dependent, constitutively activated form of Smo (SmoM2) in the mammary gland leads to paracrine stimulation of proliferation and hyperplasia [35, 36]. This pattern of SMO expression and proliferation bears a striking resemblance to proliferation patterns observed in DCIS and IBC showing ectopic expression of SMO [36]. It was hypothesized that alternative splicing analysis may offer insight into the molecular mechanisms mediating proliferative stimulation in this model system.

### Results

### Overview of DASplice

DASplice is a software framework that can take aligned single-end or paired-end reads, and perform differential alternative splicing analysis. After aligning RNA-Seq data using alignment tools such as STAR [37], DASplice tabulates the exon and splice junction information from aligned reads from each sample (i.e. experimental biological replicates). DASplice primarily uses exon boundary information from existing gene annotations such as UCSC KnownGene to accomplish this tabulation. However, DASplice also infers new splice junctions not available in existing gene annotations by identifying observed spliced reads between known exons from the raw RNA-Seq data. DASplice then builds structural gene models that are most relevant to the biological systems under study. The gene models are represented in a DAG format. This flexible data structure allows complex alternative splicing types and splicing information derived from the raw data to be represented. Thus, it is not restricted to the quality of existing gene annotations.

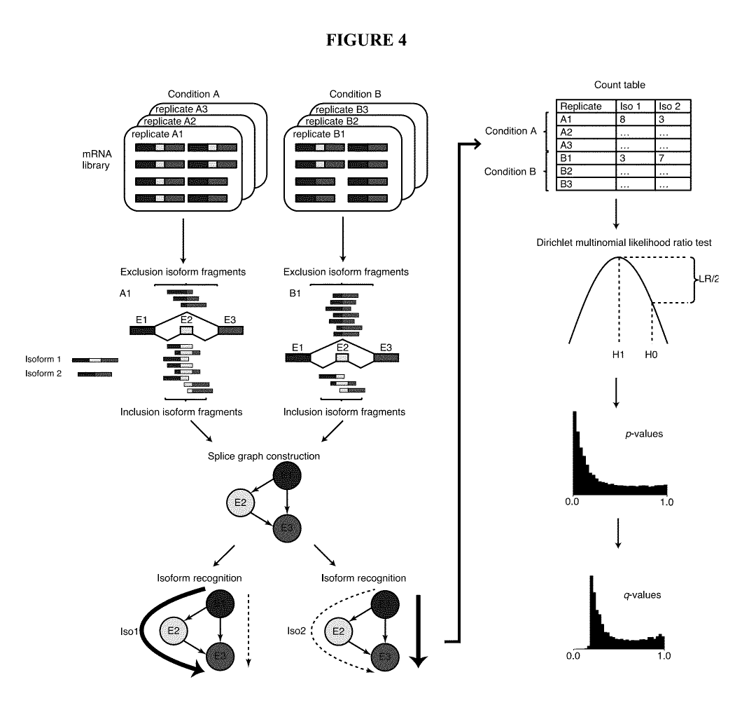

Once a gene model is built, DASplice enumerates different alternative splicing isoforms. Aligned sequence fragments are then counted in each splice isoform, thereby summarizing the observed data for a particular event across multiple biological replicates in a family of treatment conditions. DASplice models the RNA-Seq count data using the Dirichlet-multinomial (DMN) distribution [34]. To test the alternation between splice isoforms, the widely used likelihood ratio hypothesis testing (LRT) framework was employed [38]. Notably, the LRT permits analysis of arbitrary multi-treatment and multi-factorial experimental designs or studies. P-values for each alternative splicing event are computed, which are then adjusted for multiple hypothesis testing using the False Discovery Rate framework to give rise to q-values. Besides q-values, DASplice also provides log-odd-ratios for the changes in splice isoform usage between pairs of conditions or potential combinations of factor levels in a multi-treatment design. FIG. 4 displays the general workflow of DASplice.

### Simulation Analysis of the Performance of DASplice

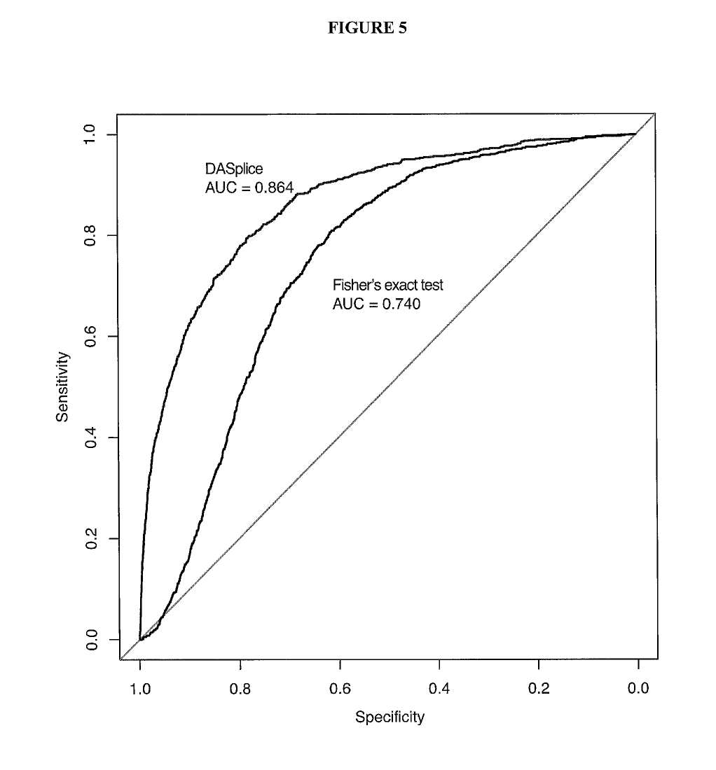

RNA-Seq data were aligned using STAR aligner and tabulated (see methods). To establish the properties of this method, first a large-scale simulation analysis was performed. The purpose of the simulation was to test the performance of DASplice compared with Fisher's exact test. This comparison was performed using simulated data generated by sampling from the RNA-Seq data from the SmoM2 mouse model. The simulation allowed an objective comparison between DASplice and Fisher's exact test given statistical parameters under control. First, a Monte Carlo method was constructed to establish simulated realizations of differential alternative splicing or its absence. Then, the variability in the count data was determined according to the simulation parameters informed by properties of the real data. A total of 10,000 test cases was simulated including both differential and non-differential alternatively spliced events as described below. To simplify the analysis, focus was placed on cassette exon events. Each test case had two genotypes and each genotype had three replications. To mimic the distribution of isoform counts, log-odds and log-odd-ratios of the test cases were randomly sampled from the real data set. To simulate overdispersion, the logarithms of the dispersion parameters were randomly sampled from a uniform (−5, 0) distribution. Given the positive correlation between overdispersion and the difficulty in calling an alternative differential splicing event, there was a requirement that the log-odd-ratio of the differentially alternative splicing event to be greater than 0.3+10.0×max(ψ₁, ψ₂), where ψ₁ and ψ₂ are the dispersion parameters for the two genotypes. Furthermore, there was a requirement that the difference of exon inclusion levels between two genotypes to be greater than 10% to classify an alternative splicing event as differential. Using this simulation scheme, a count table was generated for each test case. For both Fisher's exact test and DASplice, the sensitivities and specificities using p-values sliding from 0 to 1 were calculated. Then, the receiver operating characteristic (ROC) curves (FIG. 5) and calculated the areas under curve (AUCs) were generated. DASplice had an AUC of 0.864, which was significantly greater than Fisher's exact test (AUC=0.740, DeLong's test p=2.2×10⁻¹⁶).

### RT-PCR Analysis of the Performance of DASplice and Alternatives

Differential alternative splicing analysis were performed with DASplice using RNA-Seq data from FACS sorted mammary epithelial cells (Green and Red) derived from the SmoM2 mouse model. In this application, DASplice inferred 90 differential alternative splicing events with q<0.05 (Table 4). Events with q≧0.05 were not considered, which is common practice in RNA-Seq data analysis [39]. Since RT-PCR experiments on all the qualifying events could not be performed due to finite supplies of RNA from samples used in the primary study, a rank based strategy was used and RT-PCR experiments performed on the top ten events with the smallest q-values [40]. Nine out of ten RT-PCR assays expressed the anticipated amplicon sizes, which were then subjected to differential analysis using RT-PCR.

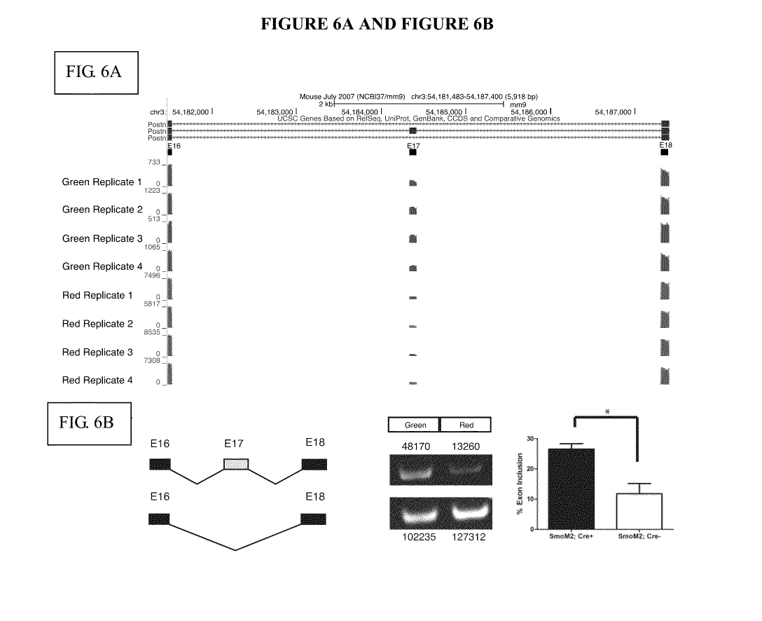

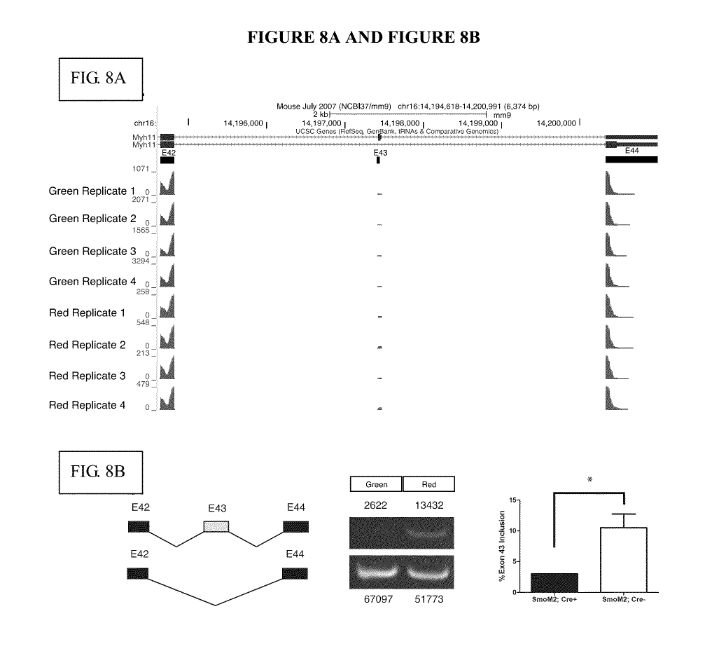

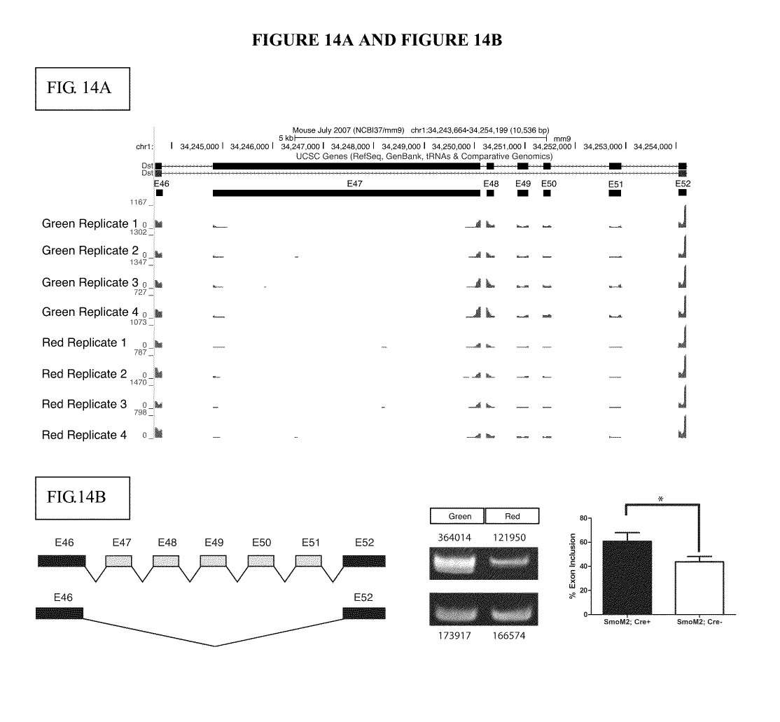

DASplice predicted that Postn, a gene associated with cancer metastasis and poor prognosis, contains an exon (E17) which is included more readily in SmoM2-expressing Green cells compared to Red SmoM2-negative cells (see Online Methods). Visualization of RNA-Seq data of this event in the UCSC genome browser tracks displayed obvious changes (FIG. 6a). This prediction was confirmed by RT-PCR where Green cells showed higher inclusion ratios of exon 17 (E17) compared to Red cells (26.5% compared to 11.75%, p<0.05) (FIG. 6b). DASplice predicted a higher frequency of inclusion of multiple cassette exons (i.e. E47-E51) in Green cells compared with Red cells (see UCSC browser tracks in FIG. 14a) in Dst, a gene that aids in the maintenance of adhesion junction integrity. PCR analysis confirmed this prediction (60.75% in Green cells compared with 43.75% in Red cells, p<0.05) (FIG. 14b). Myh11, a component of the myosin hexameric complex involved in muscle contraction, was identified by DASplice as an exon skipping event that spliced out exon 43 (E43) more frequently in Green cells compared to Red cells (see UCSC browser tracks in FIG. 8a). PCR analysis confirmed the Myh11 differential splicing prediction by DASplice (3% in Green compared to 10.5% in Red, p<0.05) (FIG. 8b).

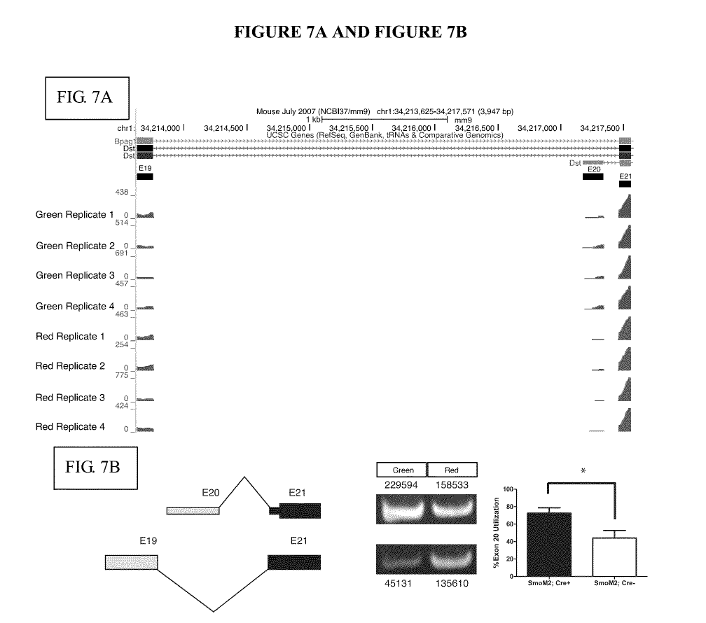

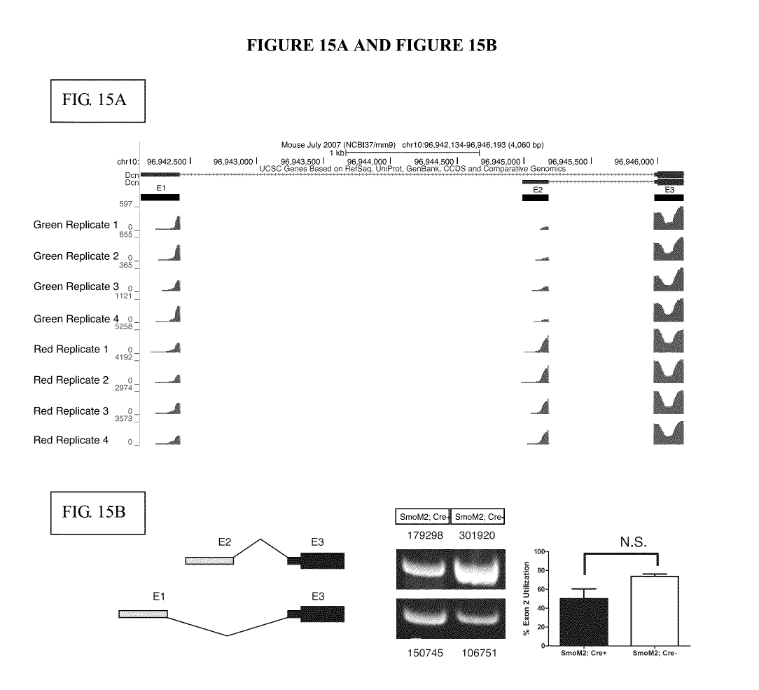

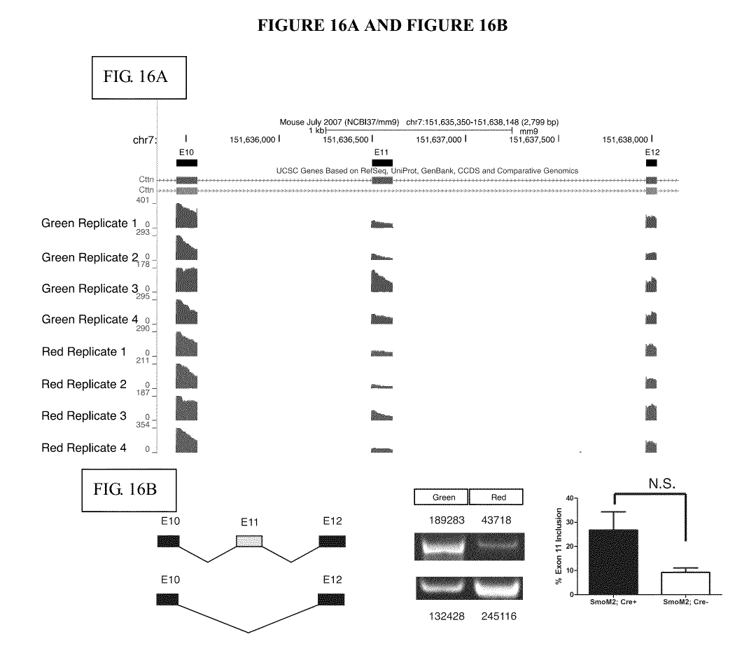

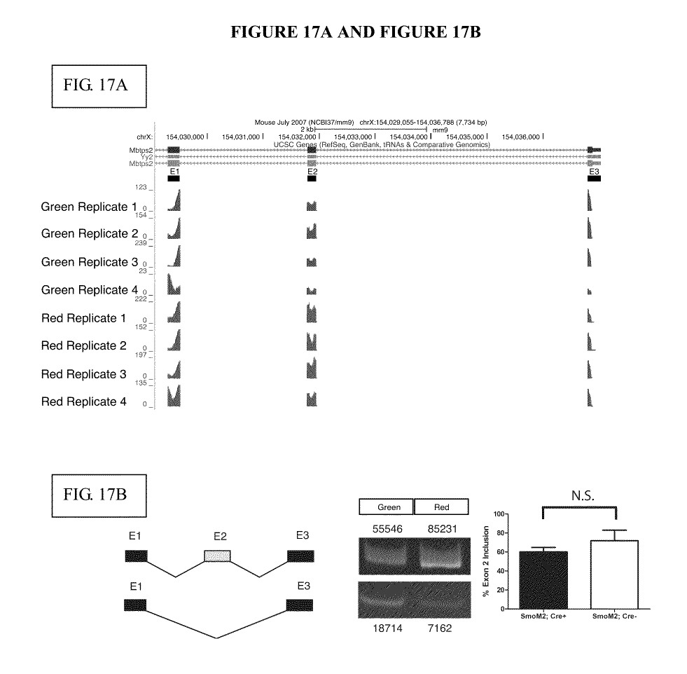

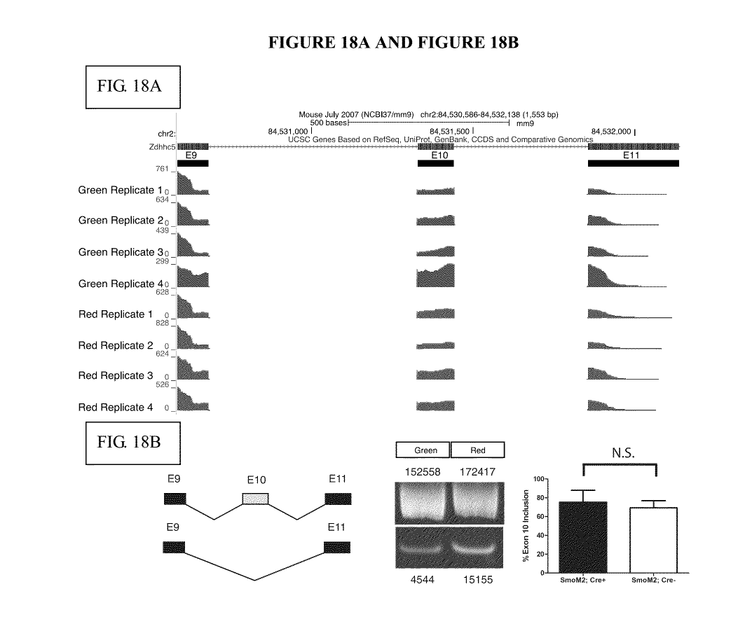

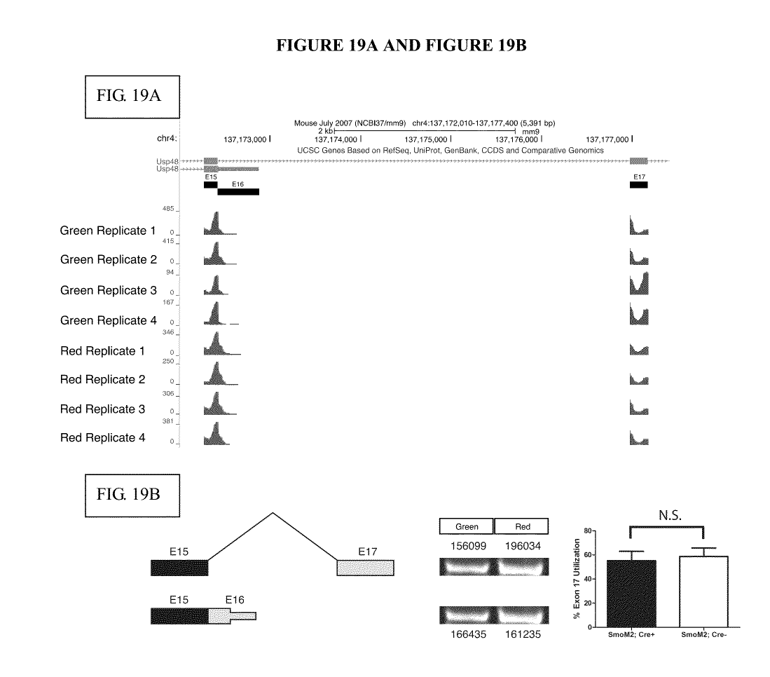

To investigate whether DASplice can also identify alternative promoter events, we designed primers against a constitutive exon (E21) and two alternating exons (E19 and E20) on Dst. Here, DASplice anticipated a higher utilization of exon 20 (E20) in Green cells (FIG. 5a) and the RT-PCR experiment demonstrated that this prediction was accurate (72.5% in Green cells compared to 44% in Red cells, p<0.05) (FIG. 7b). In the remainder of the nine events, Dcn (E1-E3), Cttn (E10-E12), Mbtps2 (E1-E3), Zdhhc5 (E9-E11) and Usp48 (E15-E17), were not confirmed by PCR analysis (p≧0.05) (FIG. 15, FIG. 16, FIG. 17, FIG. 18, and FIG. 19).

In total, four out of these nine events were were able to be confirmed. Among the four confirmed events, two (Postn (E16-E18) and Myh11 (E42-E44)) were single-cassette-exon events, one (Dst (E46-E52) was a multiple-cassette-exon event, and one (Dst (E1-E3)) was an alternative promoter event.

To test the efficiency of DASplice, it was compared with two alternative splicing analysis methods (MATS [29] and DEXSeq [31]) that are widely used for differential alternative splicing using RNA-Seq data (rna-seqblog.com). Cufflinks [41], another popular RNA-Seq data analysis method, was excluded from the comparison because it only provides differential transcript level analysis but not differential alternative splicing analysis that would allow for a head-to-head comparison. In order to have a fair comparison, MATS and DEXSeq were applied to the same data set from the SmoM2 mouse model.

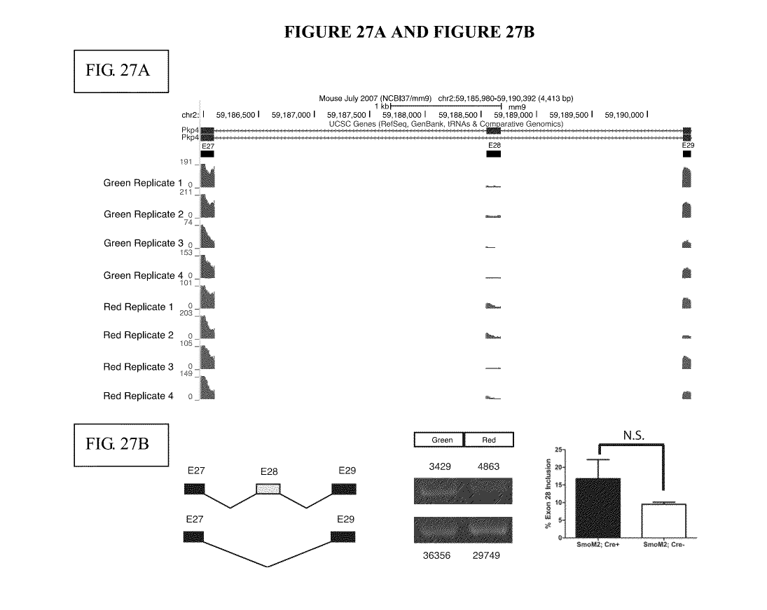

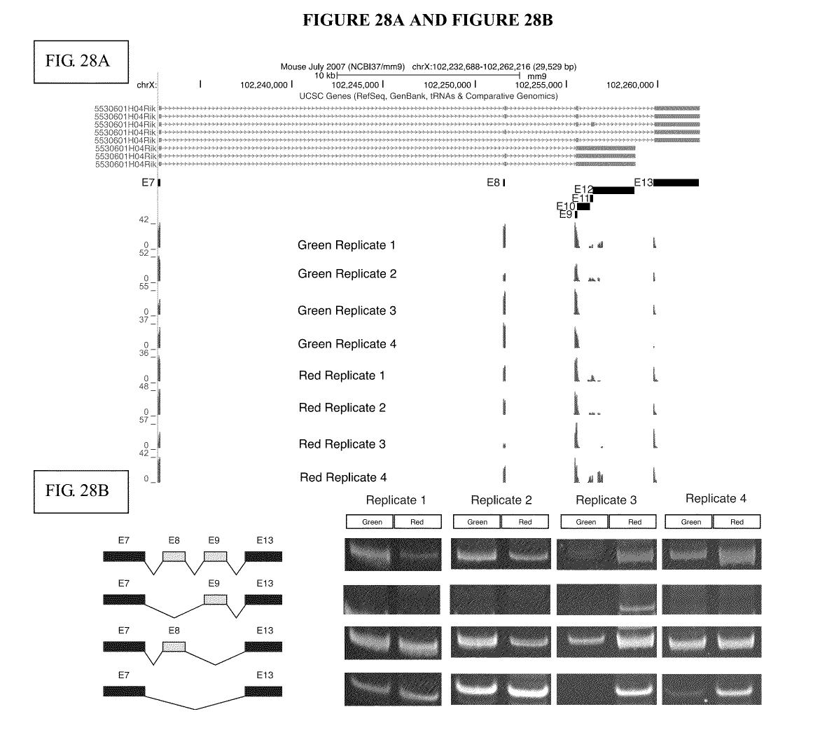

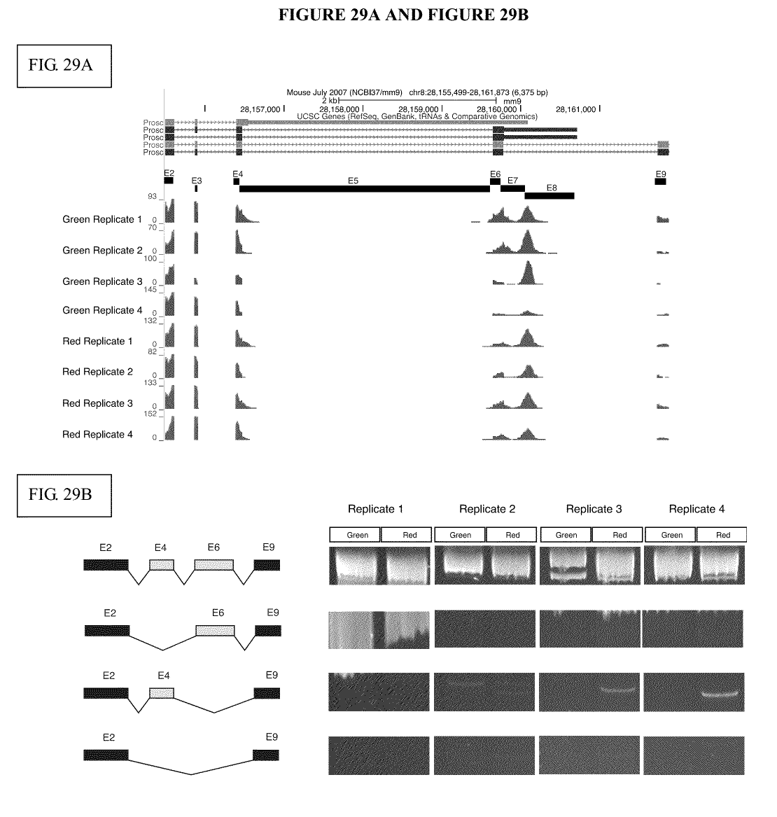

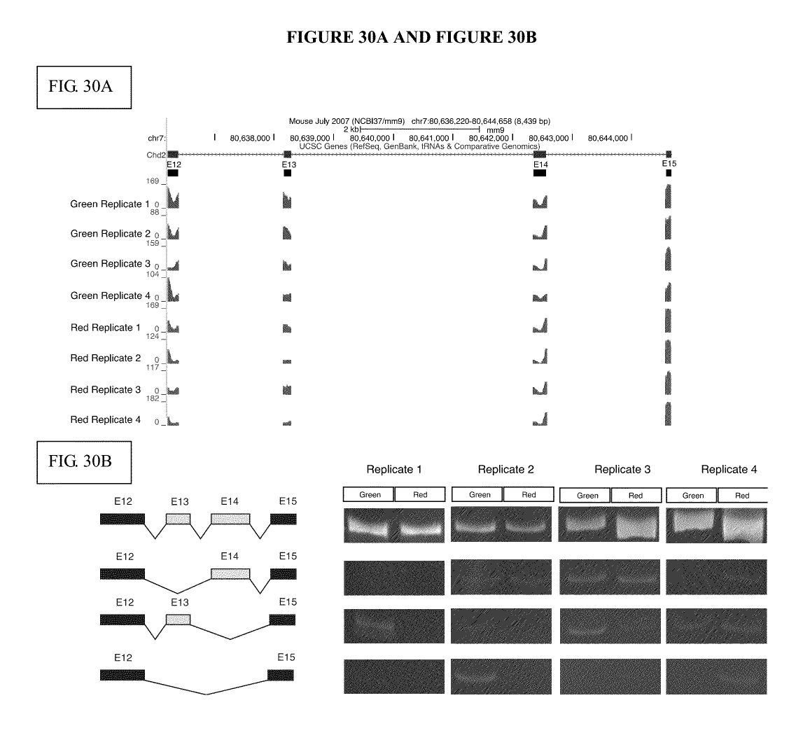

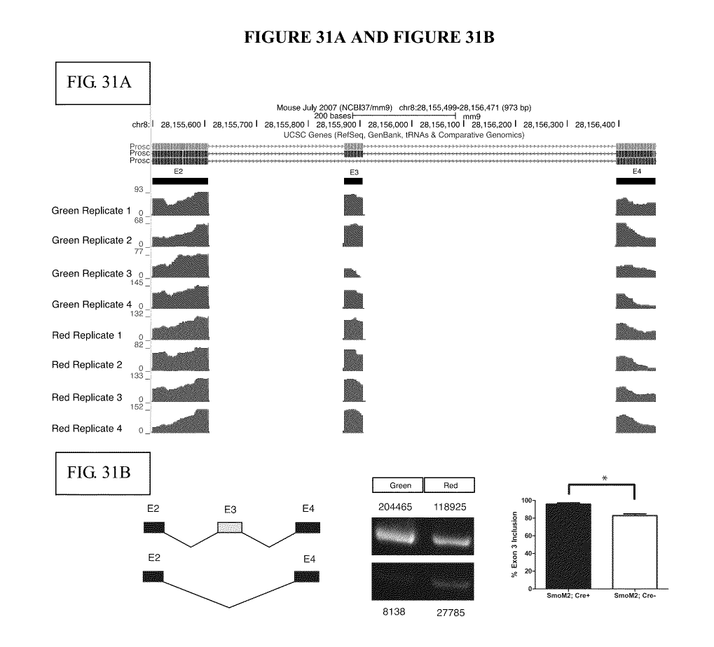

MATS identified a total of six significant differentially altered splicing events in Green vs. Red cells (Table 6, Table 7, Table 8, and Table 9) (q<0.05). MATS identified three exon skipping events and three mutually exclusive events using both exon and junction reads. Of these six events, one event, Postn (FIG. 6) was common with the results of DASplice and was the only event confirmed with RT-PCR. Pkp4 (E27-E29), an exon skipping event identified by MATS (FIG. 27a), displayed no significantly altered alternative splicing patterns as a consequence of SmoM2 expression (FIG. 27b). Prosc (E2-E4), an exon skipping event, was predicted to include the variable exon 3 (E3) more frequently in Red cells according to MATS (FIG. 31a). PCR analysis results suggest the opposite of the MATS prediction and demonstrated that E3 is more frequently included in Green cells (95.75% in Green cells compared to 82.75% in Red cells, p<0.05) (FIG. 31b). 5530601H04Rik (E7-E9, E13), Prosc (E2, E4, E6, E9), and Chd2 (E12-E15) were all classified as mutually exclusive events by MATS, a finding that was contradicted by the PCR assays (isoforms expressed in all four replicates display inclusion of both variable exons which nullifies these events as a mutually exclusive, see FIG. 28, FIG. 29, and FIG. 30). Thus, MATS only correctly predicted a single event out of six.

TABLE 1
List of primers sequences used for PCR assays.

| | Variation, | | |
|---|---|---|---|
| Sequence | direction | Primer Sequence | SEQ ID NO: |
| 5530601H04Rik | (E7-E9,E13)-F: | CACTCCAATTGGTTGTCCAG | SEQ ID NO: 1 |
| 5530601H04Rik | (E7-E9,E13)-R: | TGGAGAACAGAGGGCGACTA | SEQ ID NO: 2 |
| Chd2 | (E12-E15)-F: | TGAACTTCAACCTTTTGGTTTG | SEQ ID NO: 3 |
| Chd2 | (E12-E15)-R: | TGTGCAAGTGGATGGGACTA | SEQ ID NO: 4 |
| Cttn | (E10-E12)-F: | GATCCTTCTGCACCCCATAC | SEQ ID NO: 5 |
| Cttn | (E10-E12)-R: | AAGGCTTTGGAGGAAAGTTTG | SEQ ID NO: 6 |
| Dcn | (E1-E3)-F: | TGGCAAATACCCGGATTAAA | SEQ ID NO: 7 |
| Dcn | (E1-E3)-F2: | GCAGGAGACCCAGAATCTCA | SEQ ID NO: 8 |
| Dcn | (E1-E3)-R: | GGATTATGCCAGAAGCCTCA | SEQ ID NO: 9 |
| Dlc1 | (E018-E020)-F: | TCTGGATTCATAGCCTGCTTT | SEQ ID NO: 10 |
| Dlc1 | (E018-E020)-R: | TGAGCTACCTGGTCCTTACTCT | SEQ ID NO: 11 |
| Dst | (E1-E3)-F: | ATCCGAACGACATCGAGAAG | SEQ ID NO: 12 |
| Dst | (E1-E3)-F2: | AGTAACACCACCAGCACTCG | SEQ ID NO: 13 |
| Dst | (E1-E3)-R: | TTTGTCTTCGCAGCTGACAC | SEQ ID NO: 14 |
| Dst | (E46-E52)-F: | CTGCAGGAGGGCTACAATTT | SEQ ID NO: 15 |
| Dst | (E46-E52)-F2: | CCAAAGCAGACAACTGCTGA | SEQ ID NO: 16 |
| Dst | (E46-E52)-R: | AGGAGATCACACAGCCCTTG | SEQ ID NO: 17 |
| Mbtps2 | (E1-E3)-F: | AGAGGGAGAGTCAGCCATCA | SEQ ID NO: 18 |
| Mbtps2 | (E1-E3)-R: | ATCCGTCGCTACGATGATTC | SEQ ID NO: 19 |
| Mmp19 | (E10-E12)-F: | GAAACAAGGTGTGGCGGTAT | SEQ ID NO: 20 |
| Mmp19 | (E10-E12)-R: | TAGGGTAGCGGCTAAGGTCA | SEQ ID NO: 21 |
| Myh11 | (E42-E44)-F: | CGAGCGTCCATTTCTTCTTC | SEQ ID NO: 22 |
| Myh11 | (E42-E44)-R: | ATGAGGCCACAGAGAGCAAT | SEQ ID NO: 23 |
| Pkp4 | (E27-E29)-F: | ACACCTGTGTCCACGCTAGA | SEQ ID NO: 24 |
| Pkp4 | (E27-E29)-R: | TGTTCTCTTGCTGGTGAGGA | SEQ ID NO: 25 |
| Postn | (E16-E17)-F: | GGCAGCACCTTCAAAGAAAT | SEQ ID NO: 26 |
| Postn | (E16-E17)-R: | TCACTTCTGTCACCGTTTCG | SEQ ID NO: 27 |
| Prosc | (E2-E4)-F: | ACCTCCCAGCCATCCAAC | SEQ ID NO: 28 |
| Prosc | (E2-E4)-R: | CCATCAGTTTGTTGACGTTTT | SEQ ID NO: 29 |
| Prosc | (E2-E4,E6,E9)-F: | CAAAACCTGCAGACATGGTG | SEQ ID NO: 30 |
| Prosc | (E2-E4,E6,E9)-R: | GGTCATGAGACCCACGAACT | SEQ ID NO: 31 |
| Sprx | (E001-E003)-F: | TTGCAGCTGGGAATGCTACA | SEQ ID NO: 32 |
| Sprx | (E001-E003)-R: | AATCCATGTGAATCCCCTTGT | SEQ ID NO: 33 |
| Thoc2 | (E13-E15)-F: | TCATGTTTACTTTTTGCTTGTTTG | SEQ ID NO: 34 |
| Thoc2 | (E13-E15)-F2: | TCCTGTTTGCTTCCATCAGA | SEQ ID NO: 35 |
| Thoc2 | (E13-E15)-R: | CCTTGGTCCTCACCTCTCAC | SEQ ID NO: 36 |
| Usp48 | (E15-E17)-F: | CCAAATTCGAGGAGTGGTGT | SEQ ID NO: 37 |
| Usp48 | (E15-E17)-F2: | AATGGCAATAGTGTCGTCCTG | SEQ ID NO: 38 |
| Usp48 | (E15-E17)-R: | AACCACTTCTGCAGCCATTC | SEQ ID NO: 39 |
| Zddhc5 | (E9-E11)-F: | GAGCTGACTTGAGGCTGGAG | SEQ ID NO: 40 |
| Zddhc5 | (E9-E11)-R: | ACACACCTCAGCCTGGCTAC | SEQ ID NO: 41 |

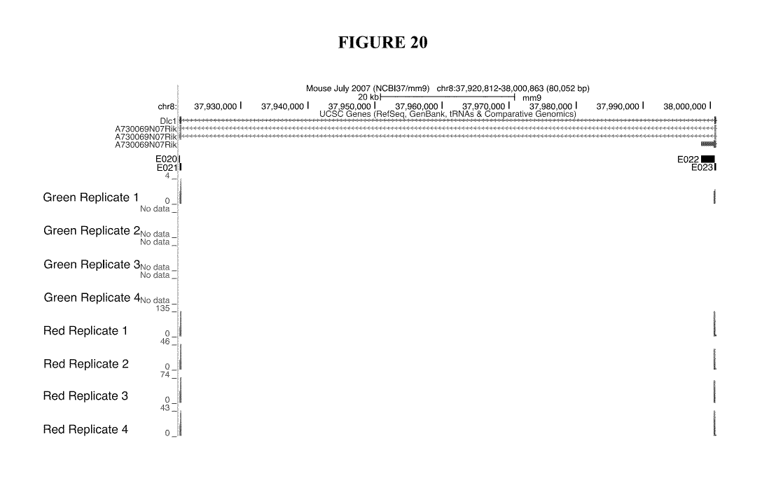

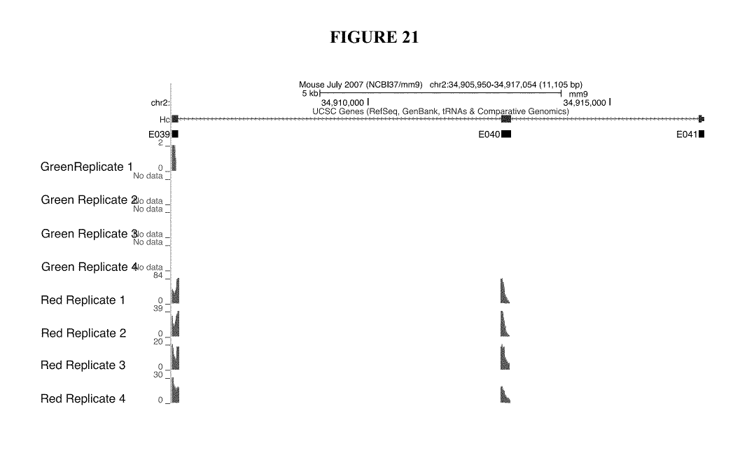

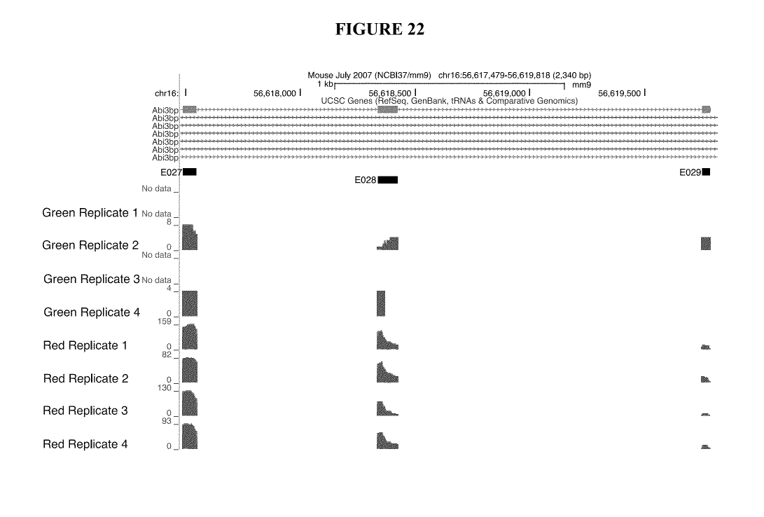

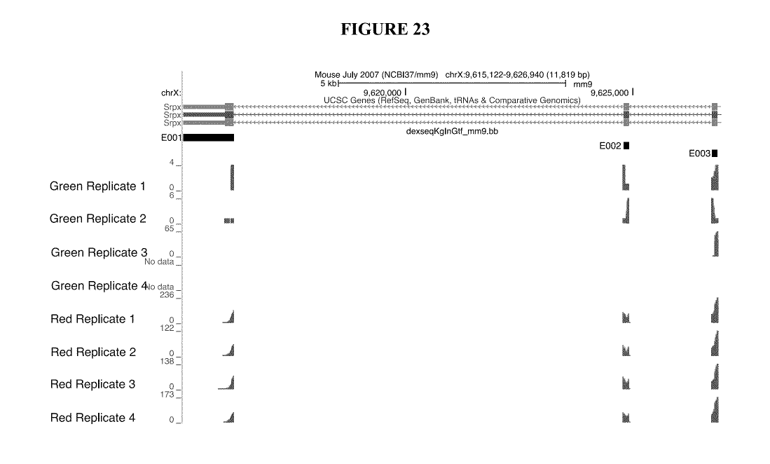

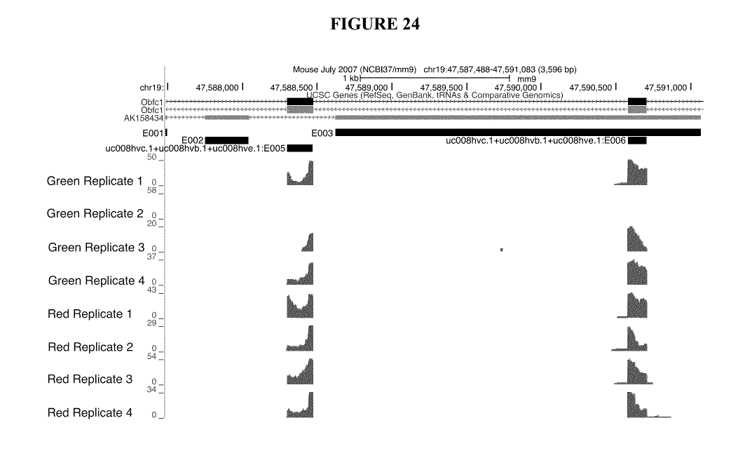

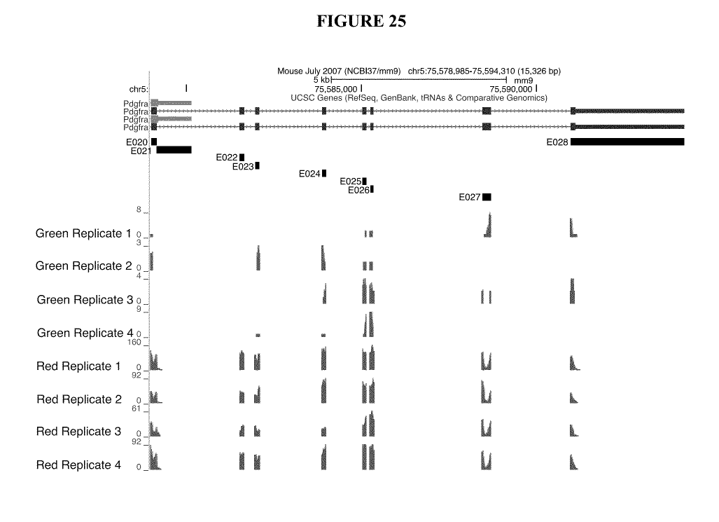

DEXSeq identified 33 events that had q<0.05 (Table 5). Unlike DASplice and MATS, DEXSeq only identifies differentially expressed exons, and does not fully annotate differential alternative splicing event types. Therefore, we had to enumerate and infer the event types by manually examining the UCSC genome browser tracks of these 33 events. It was found that the majority of the proposed events appear to have low count coverage and do not show strong visual evidence of alternative splicing (examples of tracks are provided in FIG. 20-FIG. 25). By excluding the events that do not have sufficient sequence reads on the differential exons identified by DEXSeq (Table 2), as well as the immediate flanking exons and the joining splice junctions in all biological replicates, only four events from these 33 were identified that were considered as candidates for RT-PCR experiments.

TABLE 2
Exclusion of the events that do not have sufficient sequence reads on the
differential exons identified by DEXSeq

| DEXSeq_Event_ID | Reason |
|---|---|
| A130040M12Rik_E001 | No reads in some samples |
| A830010M20Rik_E007 | Low number of reads |
| Abca8a_E019 | No reads in some samples |
| Abi3bp_E027 | No reads in some samples |
| Abi3bp_E028 | No reads in some samples |
| Abi3bp_E046 | No obvious event type |
| AK158434_E002 | No reads in some samples |
| AK158434_E003 | Low number of reads |
| Bmper_E015 | No reads in some samples |
| Col12a1_E055 | No obvious event type |
| Col15a1_E040 | No reads in some samples |
| Cxcl12_E006 | Low number of reads |
| Dlc1_E021 | No reads in some samples |
| Fbxw8_E004 | Low number of reads |
| Fmo3_E001 | No reads in some samples |
| Hc_E040 | No reads in some samples |
| Loxl2_E011 | No reads in some samples |
| Med12_E049 | Low number of reads |
| Mndal_E011 | Low number of reads |
| Nol11_E001 | Low number of reads |
| Pdgfra_E028 | Low number of reads |
| Rmst_E017 | No reads in some samples |
| Slc25a21_E001 | No reads in some samples |
| Spon1_E004 | No reads in some samples |
| Spon1_E007 | No reads in some samples |
| Srpx_E006 | Low number of reads |
| Svep_E011 | No reads in some samples |
| Usp49_E007 | Low number of reads |
| Zfp467_E002 | No reads in some samples |

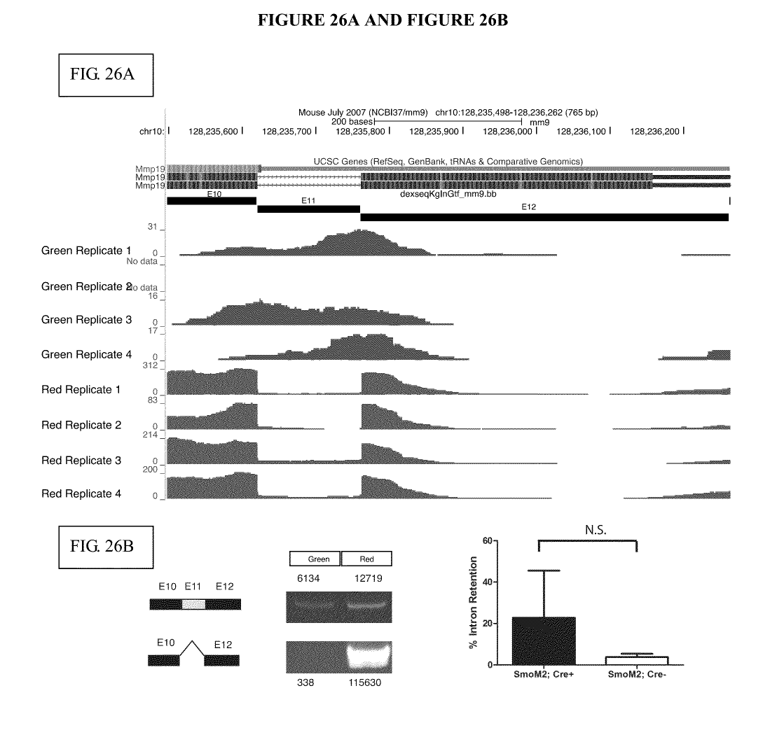

Of the four events, only two RT-PCR assays generated the anticipated amplicon sizes. One event was Mmp19 (E10-E12), which was classified as an intron retention event that was identified as having more intron retention between exons E10 and E12 in Green vs. Red cells (FIG. 26a). Upon testing the Mmp19 event, we found that neither intron-retaining or exclusion isoforms were detected by RT-PCR analysis for half of the Green replicates (FIG. 26b). Overall, the analysis of Mmp19 demonstrated that it was not significantly different between Green and Red as DEXseq predicted. The other significant event predicted by DEXSeq was Dcn, an alternative promoter event also predicted by DASplice. As has been shown, this event was not confirmed with RT-PCR analysis (FIG. 15b). Thus, it was found that none of the events identified by DEXSeq in the data produced an RT-PCR confirmed result.

In summary, DASplice identified more RT-PCR confirmed differential alternative events and had a higher confirmation rate (44.4%) than either MATS and DEXSeq using the experimental system investigated. Moreover, DASplice demonstrated the ability to identify complex alternative splicing patterns other than exon skipping events and mutually exclusive exon events. Table 3 summarizes all the events tested for DASplice, MATS and DEXSeq.

TABLE 3
Summary of RT-PCR confirmation of the predictions made by DASplice,
MATS, and DEXSeq.

| | | # Events | | |
|---|---|---|---|---|
| | # Significant | Considered for | # Events with | |
| Statistical | Events | PCR | Correct PCR | # Events |
| Method | (FDR < 0.05) | Confirmation | Products | Confirmed |
| DASplice | 90 | 10 | 9 | 4 |
| MATS | 6 | 6 | 3 | 1 |
| DEXseq | 33 | 33 | 2 | 0 |

### Discussion

Sensitive and specific analysis of RNA alternative splicing is an important methodological challenge in genomics. The growing abundance of short read RNA-Seq data encompasses the unbiased panorama of genome-wide alternative splicing. Notably, alternative splicing can be strongly differential between different biological conditions, but the nature of short-read technology permits only observation of RNA fragments rather than RNA isoforms. In the absence of true observation of isoforms, statistical methods are required to make inferences about differential alternative splicing across biological conditions. The DASplice method directly addresses this challenge.

The success of DASplice in addressing the differential alternative splicing problem is attributed to the sophisticated representation of RNA-Seq raw data, efficient utilization of prior knowledge of exon annotations to guide but not circumscribe analysis, careful modeling of biological variation, and the recent development of high performance methods for count data statistical models [34]. These characteristics enable DASplice to effectively use the information ignored by other methods, which collectively leads to more comprehensive and accurate detection of differential alternative splicing events.

Using mammary epithelial cells derived from the SMO overexpression mouse model, DASplice identified several genes that are differentially alternatively spliced and which were confirmed using RT-PCR analysis. Postn was identified by DASplice and confirmed by RT-PCR to undergo differential alternative splicing as a consequence of SmoM2 expression. This change generates different splice isoforms which can influence the function of the protein [42]. In cancer, Postn has been known to influence cell invasive and metastatic properties of different cancer cell lines depending on the specific splicing isoform that was expressed [43] and has been detected in serum from breast cancer patients that developed bone metastases [44, 45]. Dst has various splicing isoforms that appear to have different roles in the nervous system according to the different interaction domains that are available within each isoform. Myh11, a gene that is downregulated in the stroma surrounding invasive ductal carcinomas as well as in mice lacking sonic hedgehog in lung cells [46], was differentially spliced in Green compared to Red cells. Overall, these three genes play critical roles in the maintenance of cell structure as well as support of cell attachment and migration, which are all biological processes that are altered as a consequence of SMO overexpression in a recent pathway analysis (see NCBI GEO accession: GSE70210).

One distinct advantage of DASplice is its extensibility to account for multi-factorial RNA-Seq experiments. Single factorial experimental designs, where only one factor is changed (e.g., a gene is mutated), are common but restrictive forms of RNA-Seq experiments [47]. As with prior microarray technology, as the cost of RNA-Seq continues to drop, more complex experimental designs will become common [48]. For example, one may design an experiment to study the effect of a gene knockout in two or more experimentally modulated environments and apply RNA-Seq to assess the transcriptional changes. The analysis of alternative splicing for multi-factorial RNA-Seq studies necessitates a flexible statistical framework. Due to the generality of Dirichlet-multinomial (DMN) regression and the LRT approach, DASplice is applicable to multi-factorial designs and can return specific testable inferences of differential alternative splicing exploiting the full multi-factorial design [49].

Another distinct advantage of DASplice is its ability to incorporate DMN and overdispersion. DMN is a count model that fundamentally utilizes the discrete nature of deep sequencing data, allowing analysis to proceed in situations where counts are low or zero in some experimental replicates. Importantly, the DMN model considers overdispersion, so that replicates within a treatment are allowed to vary by more than would be expected under the simpler multinomial model of category counts (e.g., the multinomial model as used in Fisher's exact tests and related extensions).

A third advantage of DASplice is its ability to consider arbitrary alternative splicing isoforms. As sequencing cost continues to drop, it becomes easier to sequence the transcriptome more deeply. The number of splicing events is, in theory, combinatorially unlimited [27]. The conventional exon centric view will not be sufficient [31, 50] to comprehensively characterize all possible alternative splicing events. The flexible DAG data structure offered by DASplice is an important alternative to conceptualize and represent splicing data. Much like the SAM format is a de facto standard for sequence alignment due to its flexibility to store alignment information generated by various aligners [51], a data format based on the DAG data structure can be a substrate for future standardization of alternative splicing analysis results.

In conclusion, DASplice represents an important milestone to potentiate genome wide discovery of differential alternative splicing in various experimental conditions, tissues, cell types and disease states. This tool can lead to improved understanding of the complexity arising from alternative splicing in diverse contexts. It should be noted that despite the contribution that has been made with DASplice, the results indicate that differential alternative splicing analysis in short read RNA-Seq data remains a challenging problem, especially for experiments with a small number of samples or experiments with large variability. There may be scenarios where other methods can perform better than DASplice. For instance, because DEXSeq is designed with exon usage as the primary consideration, DEXSeq might perform well for a special data set where there are only differential exon skipping events or where biological interest is specifically focused on this class of events. However, since DASplice is a more general framework, it may be expected that further methodological research using DASplice combined with the strengths of other methods will lead to more powerful and broadly applicable tools for this important biological problem.

### Mouse Genetics

### Mouse Strains

Mouse lines Gt(ROSA)26Sor^{tm1(Smo/YFP)Amc}/J, carrying a Cre-dependent knock-in allele of SmoM2 at the ROSA26 locus (herein designated SmoM2^{c}), and Gt(ROSA)26Sor^{tm4(ACTB-tdTomato,-EGFP)Luo}/J, carrying the mT-mG dual fluorescence Cre recombinase reporter targeted to the ROSA26 locus (herein designated mT-mG) were obtained from Jackson laboratories (stock numbers 005130 & 007576, respectively). Lines were maintained as homozygotes in the original 129X1/SvJ, C57BL/6, and Swiss Webster mixed strain (SmoM2^{c} mice) or in a mixed genetic background (mT-mG mice). The MMTV-Cre [52] line (expressing Cre recombination under the control of the mammary selective Mouse Mammary Tumor Virus (MMTV) promoter) was maintained as a hemizygous transgene in a mixed genetic background. Genotyping for all alleles was carried out by allele-specific PCR using the following primers: mT-mG: Forward-CTCTGCTGCCTCCTGGCTTCT [SEQ ID NO: 42], R1-CGAGGCGGATCACAAGCAATA [SEQ ID NO: 43], R2-TCAATGGGCGGGGGTCGTT [SEQ ID NO: 44]. SmoM2: F-TGGTCTCCAACCCATTCTGC [SEQ ID NO: 45], R-GAACTTGTGGCCGTTTACGTCG [SEQ ID NO: 46]. Cre: F-AAACGTTGAATCCGAAAAGA [SEQ ID NO: 47], R-ATCCAGCTTACGGATATAGT [SEQ ID NO: 48].

### Mouse Breeding

MMTV-Cre mice were crossed with the mT-mG reporter line, and offspring intercrossed to generate males homozygous for mT-mG and hemizygous for MMTV-Cre for use in subsequent crosses. Experimental mice expressing MMTV-Cre and WT littermates (mT-mG/SmoM2^{c}; MMTV-Cre and mT-mG/SmoM2^{c}; +) were generated by crossing SmoM2^{c} homozygous females to males homozygous for mT-mG and hemizygous for the MMTV-Cre transgene. This system allowed the use of EGFP as a surrogate for SmoM2-expressing cells (hereinafter referred to as “Green”). Surrounding cells that did not undergo Cre-mediated recombination lacked SmoM2 expression and remained labeled with tdTomato Red (hereinafter referred to as “Red”). All animals were maintained in accordance with the NIH guide for the care and use of experimental animals.

### FACS Sorting and RNA Isolation.

Mammary Epithelial Cells (MECs) from 9 week old mT-mG/SmoM2^{c}; MMTV-Cre and mT-mG/SmoM2^{c}; + mice were freshly isolated using #1, #3, #4 (minus lymph node) and #5 mammary glands. The glands were then minced into 1 mm³ fragments using a Vibratome Series 800-McIlwain Tissue Chopper (Vibratome, St. Louis, Mo.) and digested in 2 mg/ml collagenase A (Roched Applied Scence, Indianapolis, Ind.) in F12 Nutrient Mixture containing antibiotic-antimycotic (Invitrogen, Carlsbad, Calif.) for one hour at 37° C. with shaking at 200 rpm. Single cells were purified as described previously [53]. Single cells were suspended in HBSS supplemented with 2% FBS and 10 mM HEPES, passed through a 40 μm filter strainer, and stained with a lineage panel cocktail to exclude non-epithelial cells as previously described [54]. Cells in which SmoM2^{c} is activated by Cre-mediated recombination express Cre-dependent EGFP, while cells in which SmoM2^{c} is not activated by recombination remain tdTomato positive. mT-mG/SmoM2^{c}; MMTV-Cre (tdTomato) and mT-mG/SmoM2^{c}; MMTV-Cre (EGFP) MECs were sorted into Red, and Green populations by FACS using an Ariall instrument (BD Biosciences). Total RNA was isolated from the different MEC populations using a Qiagen miRNeasy kit according to manufacturer's instructions (Qiagen). cDNA was synthesized using Nugen's WT Ovation kit per manufacturer's instructions.

### RT-PCR Experiment and Quantification Analysis of Splicing Events.

PCR primers were designed complementary to the constitutive exonic region flanking the predicted alternative splicing isoforms using Primer3Plus. Design of each assay was confirmed by using the UCSC genome browser in-silico PCR tool. RT-PCR was then performed on each sample replicate using selected primers for every selected gene. Each amplicon was then separated using PAGE, followed by ethidium bromide staining, imaging, and molecular weight-corrected quantification using the Kodak Gel Logic 2200 and Molecular Imaging Software. Percent inclusion was quantitated by adjusting band intensity for length of the RT-PCR product, dividing the intensity of the band which entailed the regulated alternative region by the total product. Differences in alternative splicing between Green cells and Red cells were assessed using two-sided paired Student's t-test (n=4 for Green and n=4 for Red).

### RNA-Seq Library Preparation.

Purified double-stranded cDNA is generated from 10 ng of total RNA and amplified using both 3′ poly(A) selection and random priming throughout the transcriptome. 3 mg of each sample was sheared using the Covaris S2 focused-ultrasonicator following the NuGEN Encore NGS Library System 1 protocol to obtain final library insert size of 150-200 bp. A double-stranded DNA library was created using 200 ng of sheared, double-stranded cDNA, preparing the fragments for hypbridization onto a flowcell. This was achieved by first creating blunt ended fragments ligating unique adapters to the ends. The ligated products were amplified using 15 cycles of PCR. The resulting libraries were quantitated using the NanoDrop ND-1000 spectrophotometer. Library fragment size was assessed using the Agilent Bioanalyzer DNA 1000 chip. A qPCR quantitation was performed on the libraries to determine the concentration of adapter ligated fragments using a Bio-Rad iCycler iQ Real-Time PCR Detection System and KAPA Library Quant Kit.

### RNA-Seq Raw Reads Alignment and Annotation.

The raw paired-end RNA-Seq reads were aligned to mouse (mm9) genomes using STAR with default settings and filtered for uniquely mapped reads. The number of reads for each exon and each exon-exon junction in each RNA-Seq file were computed by using the Python package HTSeq [55] using the annotation of the UCSC KnownGene (mm9) annotation [56]. Following alignment, a gene-level QC analysis was performed using hierarchical clustering. Samples that were extreme outliers from their respective treatment groups were excluded from subsequent analysis.

### Directed Acyclic Graph (DAG) Representation of Complex Alternative Splicing Types.

Due to the complexity found in different types of alternative splicing, a data structure that is flexible and capable in representing various splicing types was employed. A graph in the mathematical sense [57] (i.e., a set of objects or nodes with links called edges between some pairs) is ideal for representing complex alternative splicing events [58]. More specifically, a notation for splicing analysis using a directed acyclic graph (DAG) was designed [57]. A requirement of nodes in a DAG is that they should be partially ordered. This can be naturally determined because exons are ordered by chromosomal coordinates and the known direction of transcription of a gene.

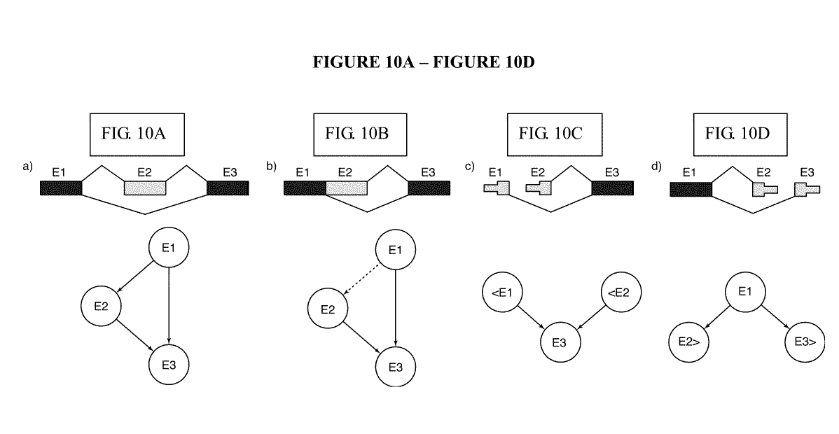

To better explain this graphical notation, three examples of common splicing patterns and alternating promoter usage are present (See FIG. 10a-c). In this depiction, an exon is represented by a node and a splice junction is represented by an edge, as demonstrated in FIG. 10a. When a splice site is harbored in an exon, the long exon is broken into exonic parts (also represented by nodes). Since the exonic parts are adjacent to each other, the two nodes will be connected by a dashed edge as shown in FIG. 10b. Alternative promoter events in the 5′ end of a gene and alternative poly-A in the 3′ end of a gene are indicated by “<” and “>” respectively as illustrated in FIG. 10c.

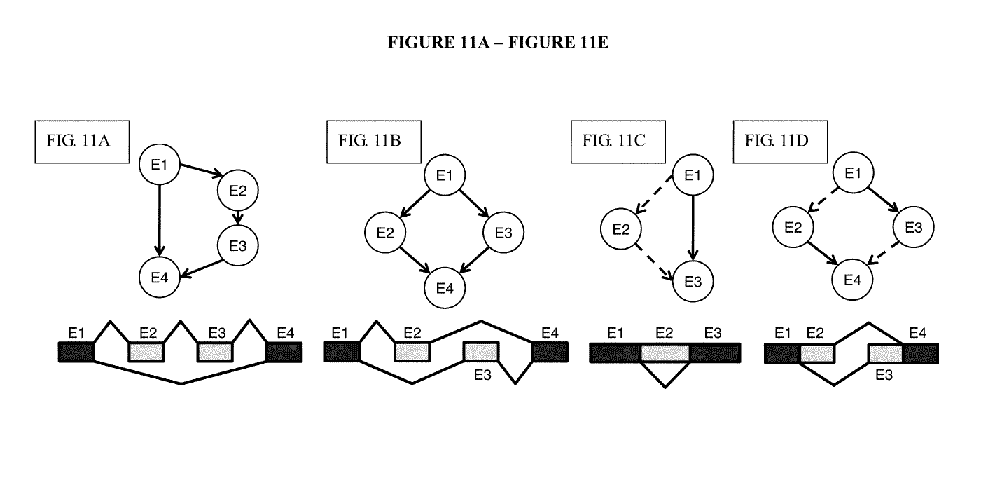

With this notation, a complex alternative splicing type can be translated into its corresponding graph representation. At the same time, isoforms associated with a splicing type can be identified from the graph. FIG. 11a-d provides four examples of such translation between the DAG representation and particular splicing events.

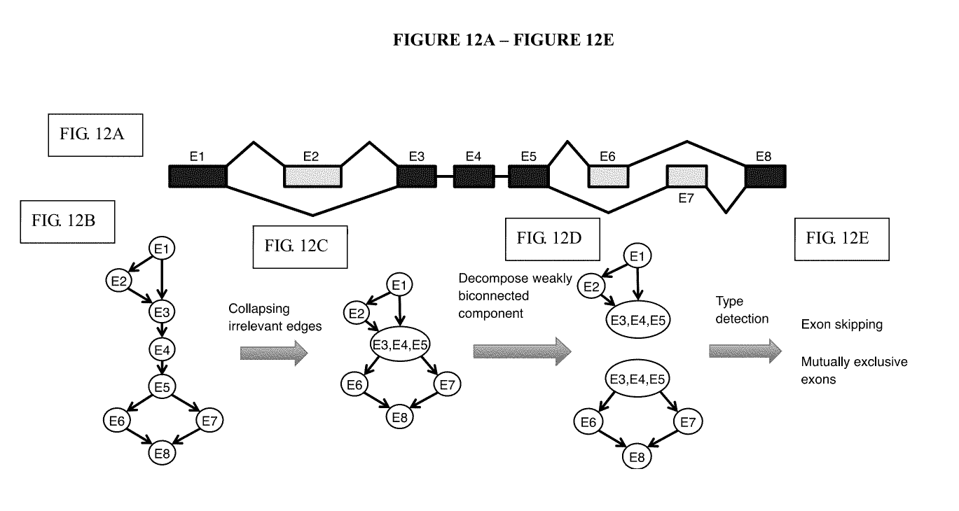

A gene typically has many exons, for example, FIG. 12a shows a gene with a skipped exon E2 and two mutually exclusive exons E6 and E7. Although such alternative splicing types can be easily visualized, a graph algorithm to automate such detection was designed as described below.

A gene model can be first converted into a graph representation as described above in FIG. 12b. Since the path E3-E4-E5 does not harbor an alternative splicing event, the edges on this path can be collapsed to result in a pseudo-node “E3,E4,E5”. In essence, nodes in any path without a branch should be collapsed into a pseudo-node. After collapsing the nodes, the result is a summary as presented in FIG. 12c. This example presents the basic structures of an exon skipping event and a mutually exclusive exon event. To automatically detect such structures, a graph algorithm to decompose the splicing DAG was designed. The decomposed graphs are called weakly biconnected components [59]. Then, these decomposed components can be readily classified into different alternative splicing types as described in the previous subsection.

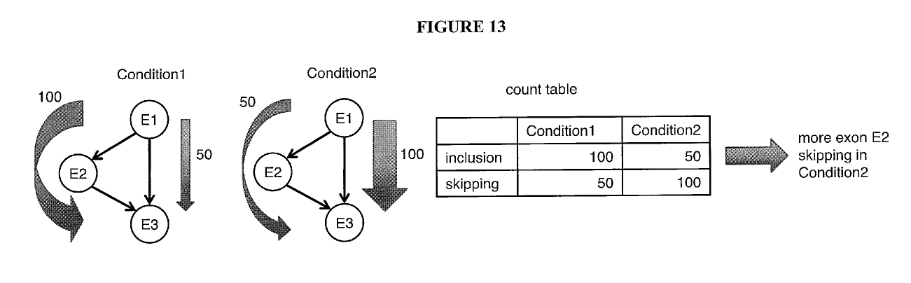

Once the alternative splicing events are translated into the graph forms, splicing isoform information can be extracted as shown in FIG. 13. The number of raw RNA-Seq reads that are uniquely mapped to each of the isoforms can be summarized in a count table.

### Statistical Inference of RNA-Seq Data Using Count Data Statistics

There are a number of statistical models available for count data analysis [60, 61]. However, to perform statistical hypothesis testing based on count tables generated from RNA-Seq experiments, the Dirichlet-multinomial distribution was found to be one of the most appropriate statistical models.

To model the switching between two splicing isoforms, one can assume that reads are randomly distributed to the two isoforms according to the binomial distribution. When there is switching between more than two splicing isoforms, a more appropriate statistical model is the multinomial distribution, which can have any number of categories. The multinomial distribution is determined by a total count parameter and a set of proportion parameters indicating the probability of each trial (e.g. read) falling into each category. The variance of the multinomial distribution is uniquely determined once the proportion parameters are established. However, it is well known that real RNA-Seq data sets exhibit larger variance [39, 62], rendering the multinomial (or binomial) distribution inappropriate for modeling real biological data. The DMN distribution extends the multinomial distribution by having an additional dispersion parameter making it an ideal model for differential alternative splicing analysis.

Due to the importance of the DMN distribution in modeling high-throughput sequencing data, a new algorithm was recently developed that dramatically increases the runtime and improves the accuracy of DMN log-likelihood function computation [34]. Based on this distribution, an efficient and novel regression procedure was developed. The DMN regression procedure resulted in a software that provides a flexible framework for hypothesis testing based on the widely used likelihood ratio test [38], and as an application, this statistical framework allows efficient testing of differential alternative splicing.

### Details Regarding the Dirichlet-Multinomial Distribution

The Dirichlet-multinomial (DMN) distribution [63] is a count-data distribution that models how a given number (N) of objects are allocated to a given number (K) of categories. This distribution extends the commonly known multinomial (MN) distribution by allowing the modeling of a phenomenon called overdispersion [49]. Overdispersion refers to the situation that the actual variation is greater than the nominal variance of a distribution. A commonly employed distribution extending the Poisson model is the negative-binomial distribution [64], which results from treating observations as arising from a compound process where Gamma distributed rate parameters are sampled and conditionally generate Poisson distributed observations. This distribution is commonly used in high-throughput sequencing data analysis [39, 62].

The MN distribution is first described since it is the basis of the DMN distribution. The probability mass function (PMF) of an observation x=x₁, . . . , x_{K} following a MN distribution with K categories and N independent trials can be written as

$${f}_{MN} x ; N , p = \frac{N !}{{\sum }_{k = 1}^{K} {x}_{k} !} {\sum }_{k = 1}^{K} {p}_{x}^{{x}_{k}} ,$$

where p=(p₁, . . . , p_{K}) are the parameters of the probability that the K categories occur (ᵢ₌₁^{K}pᵢ=1). The sum of all the xᵢ's equals N (ᵢ₌₁^{K}xᵢ=N) because each trial must be in one of the K categories, and each xᵢ is a non-negative integer.

The DMN distribution is generated by allowing p to take a prior distribution conjugate to the PMF of the MN distribution f_{MN}(x; N, p), a.k.a. the Dirichlet distribution [65]

$${f}_{Dir} p ; α = \frac{Γ ( A )}{{\sum }_{k = 1}^{K} Γ ( {α}_{i} )} {\sum }_{i = 1}^{K} {p}_{i}^{{α}_{i} - 1} ,$$

where Γ x is the gamma function and A=ᵢ₌₁^{K}αᵢ. By integrating p, the PMF of the DMN distribution [66] can be written as

$${f}_{DMN} x ; N , α = \frac{N !}{{\sum }_{k = 1}^{K} {x}_{k} !} \frac{Γ ( A )}{Γ ( A + N )} {\sum }_{k = 1}^{K} \frac{Γ ( {α}_{k} + {x}_{k} )}{Γ ( {α}_{k} )} .$$

Note that the difference between the DMN distribution and the MN distribution is that p in the MN distribution is replaced by α in the DMN distribution. It is common to reparameterize α as p/ψ, where ψ=1/A is the dispersion parameter and p=a/A is the proportion parameter. Using this parameterization, the likelihood function of the DMN distribution can be written as

[symbol]

p, ψ; x=f_{DMN} x; N, α,

where N is omitted on the left hand side as it is just the sum of all the elements in the observation x.

Due to the Γ functions in the DMN likelihood function, it is difficult to directly compute the likelihood function accurately and efficiently. A new mesh algorithm that dramatically increased the speed of the likelihood function numerical computation with simultaneous improvement in accuracy was recently developed [34]. This new method can be used to efficiently evaluate DMN regressions.

### DMN Regression

Using the DMN distribution, a new regression method for differential alternative splicing analysis was developed. The details are described in this section.

Let the function l(θ, ψ; y) be the log-likelihood function of an observation of a K-category DMN random variable y, where θ and ψ and are the proportion and dispersion parameters of the DMN distribution, respectively. Note that y is a length-K nonnegative integer row vector, θ is a length-K nonnegative row vector with all of its elements summed to 1, and ψ is a nonnegative scalar.

Suppose that one has N observations of DMN response variables yᵢ (i=1, . . . , N), and each observation has a corresponding proportion parameter θᵢ and corresponding dispersion parameter ψᵢ. The total log-likelihood for all observations y can be written as

$$L θ , ψ ; y = {\sum }_{i = 1}^{N} l {θ}_{i} , {ψ}_{i} ; {y}_{i} , \\ where$$

$${y}_{N \times K} = \begin{matrix}{y}_{1} \\ ⋮ \\ {y}_{N}\end{matrix} , \\ {θ}_{N \times K} = \begin{matrix}{θ}_{1} \\ ⋮ \\ {θ}_{N}\end{matrix} , \\ and$$

$${ψ}_{N \times 1} = \begin{matrix}{ψ}_{1} \\ ⋮ \\ {ψ}_{N}\end{matrix} .$$

The product term beneath a symbol denotes the dimension of the matrix represented by the symbol. For example, N×K undery indicates that y is a N-by-K matrix.

Let η_{θ}(·) be the link function (invertible by definition) [49] for all θᵢ, and let its corresponding linear predictor (to be described below) for the ith observation be

$${{( {η}_{θ} )}_{i}}_{1 \times ( K - 1 )} = {η}_{θ} {θ}_{i} .$$

Note that the number of elements of (η_{θ})ᵢ is one less than the number of elements of θᵢ, because there is the constraint that all the elements of θᵢ sum to 1.

When there are K categories, it is customary to use the baseline-category logits model for the proportion parameter θᵢ [60]. The elements of the row vector θᵢ can be written explicitly as θᵢ=(θᵢ₁, . . . , θ_{iK}). The baseline-category logits model specify the link function such that the elements of the row vector (η_{θ})ᵢ follow

$${( {η}_{θ} )}_{ik} = ln \frac{{θ}_{ik}}{{θ}_{ik}} , \\ for$$

$$k = 1 , 2 , … , K - 1.$$

Similarly, let η_{ψ}(·) be the link function for all ψᵢ, and let its corresponding linear predictor for the ith observation be

$${{( {η}_{ψ} )}_{i}}_{1 \times 1} = {η}_{ψ} {ψ}_{i} .$$

As shorthand notations, let

$${{η}_{θ}}_{N \times K - 1} = \begin{matrix}{η}_{θ1} \\ ⋮ \\ {η}_{θ N}\end{matrix}$$

$$and$$

$${{η}_{ψ}}_{N \times 1} = \begin{matrix}{η}_{{ψ}_{1}} \\ ⋮ \\ {η}_{{ψ}_{N}}\end{matrix} .$$

Following the Generalized Linear Model (GLM) framework [49], the predictors η_{θ} and η_{ψ} are linearly related with their corresponding regression coefficients β_{θ} and β_{ψ} by

$${η}_{θ} = {{X}_{θ}}_{N \times {p}_{θ}} {{β}_{θ}}_{{p}_{θ} \times ( K - 1 )}$$

$$and$$

$${η}_{ψ} = {{X}_{ψ}}_{N \times {p}_{ψ}} {{β}_{ψ}}_{{p}_{ψ} \times 1} ,$$

where X_{θ} and X_{ψ} are the corresponding design matrices whose sizes are N×p_{θ} and N×p_{ψ}, respectively.

Therefore, the log-likelihood function can be reparameterized as

L θ, ψ; y=L β_{θ}, β_{ψ}; y.

The DMN regression problem is a maximum-likelihood problem, i.e., the problem to optimize β_{θ} and β_{ψ} as in

β_{θ}, β_{ψ}=argmax_{β}_{_{θ}}_{,β}_{_{ψ}}L β_{θ}, β_{ψ}; y.

Then one can use likelihood ratio test [38] to test the whether there are any significant changes in the proportion θ between biological conditions. For example, suppose that one has two genotypes G1 and G2, and suppose that the proportion parameters are θ₁ and θ₂. Then, one can form two hypotheses H₀ and H₁:

H0: θ₁=θ₂

H1: θ₁≠θ₂.

The ratio of the likelihoods evaluated at the MLEs under these two hypotheses can be computed, and then the significance is tested using χ² statistics.

### Correction for Multiple Hypothesis Testing.

False discovery rates (FDR) were calculated using the Benjamini-Hochberg method [67].

TABLE 4
DASplice

| EventID | RminusGlogodds | pval | qval |
|---|---|---|---|
| uc008pfh.2,uc008pfi.2,uc008pfj.2:chr3:+:54165028:54194963: | 1.342721324 | 2.93E−35 | 9.12E−32 |
| Postn:16:18-19 | | | |
| uc007aof.1,uc007aog.1,uc007aoh.1,uc007aoi.1,uc007aoj.1,uc007aok.1, | 1.279240616 | 1.43E−11 | 2.22E−08 |
| uc007aol.1,uc007aom.1,uc007aon.1,uc007aoo.1, | | | |
| uc007aop.1:chr1:+:33965069:34365497:Bpag1,Dst:44-45-46: | | | |
| 52-53-54-55-56-57-58-59-60-61-63-64-65-66-67-68-69-70-71-72-73-74> | | | |
| uc007aof.1,uc007aog.1,uc007aoh.1,uc007aoi.1,uc007aoj.1,uc007aok.1, | −1.543057276 | 1.20E−08 | 1.24E−05 |
| uc007aol.1,uc007aom.1,uc007aon.1,uc007aoo.1, | | | |
| uc007aop.1:chr1:+:33965069:34365497:Bpag1,Dst::21-22-23> | | | |
| uc007yhd.2,uc007yhe.2,uc007yhf.2,uc007yhg.2:chr16:−:14194619: | −1.436458381 | 1.00E−07 | 7.78E−05 |
| 14291501:Myh11,mKIAA0866:<2-3-4-5-8-9-10-11-12-13- | | | |
| 14-15-16-17-18-19-20-21-22-23-24-25-26-27-28-29-30-31- | | | |
| 32-33-34-35-36-37-38-39-40-41-42:44> | | | |
| uc007gwx.2,uc007gwy.2:chr10:+:96942133:96980796:Dcn::3- | −1.957549515 | 4.02E−07 | 0.0002163 |
| 4-5-6-7-8-9> | | | |
| uc009kqh.1,uc009kqi.1:chr7:−:151621628:151656646:Cttn:8- | 1.183931891 | 5.05E−07 | 0.0002241 |
| 9-10:12-13-14-15-16-17-18> | | | |
| uc009usa.1,uc009usb.1,uc009usc.1,uc012hra.1:chrX:−:153973302: | −1.747796032 | 7.99E−07 | 0.0002481 |
| 154036647:Mbtps2,Yy2:<1:3-4-5-12-13-14-15-16-17-18> | | | |
| uc009tao.1,uc009tap.1,uc009taq.1:chrX:−:39148170:39265078: | 1.248511539 | 1.14E−06 | 0.0003219 |
| BC005561,Thoc2:13: | | | |
| uc008vjh.1,uc008vji.1,uc008vjj.1,uc008vjk.1,uc008vjl.1,uc012dnk.1, | 1.128541769 | 1.36E−06 | 0.0003349 |
| uc012dnl.1:chr4:+:137149666:137214452:Usp48:14-15: | | | |
| uc008kiz.2:chr2:−:84528076:84555321:Zdhhc5:<2-3-4-5-6-7-8-9:11-12> | 2.352977706 | 1.40E−06 | 0.0003349 |
| uc008jtr.1,uc008jts.1,uc008jtt.1,uc008jtu.1,uc008jtv.1,uc012bvt.1: | 1.405339099 | 2.07E−06 | 0.0004588 |
| chr2:−:59737419:59963867:Baz2b:15-16-17-18-19-20-21- | | | |
| 22:24-25-26-27-28-29-30-31-32-33-34-35-36 | | | |
| uc009jhw.2,uc012fsk.1:chr7:−:121190295:121261295:Rras2::6-7> | 2.225218221 | 2.64E−06 | 0.0005477 |
| uc008jip.1,uc008jiq.1,uc008jir.1,uc012bug.1:chr2:−:34532501: | −1.444456615 | 4.44E−06 | 0.0008110 |
| 34610752:Gapvdl:<2-3-4-5: | | | |
| uc007rqb.1,uc007rqc.1,uc007rqd.1,uc011zdo.1:chr13:−:100787948: | −1.343975528 | 8.35E−06 | 0.0013215 |
| 100874025:Bdp1,mKIAA1241:<1-2-3-4-5-6-7:9-10-11- | | | |
| 12-13-14-15-16-17-18-19-20-21 | | | |
| uc008dko.1,uc008dkp.1,uc008dkq.1,uc008dkr.1,uc008dks.2, | 1.285153689 | 8.51E−06 | 0.0013215 |
| uc012awk.1,uc012awl.1:chr17:+:69506149:69639327:Epb4.1I3: | | | |
| <13-15-18-20-21-22-23:25> | | | |
| uc007yzr.1,uc007yzs.1,uc012aet.1,uc012aeu.1:chr16:+:32914185: | 1.339537729 | 1.19E−05 | 0.0017624 |
| 33016115:Lrch3:<2-3-4-5-6-7-8-9-10-11-12-13: | | | |
| uc009bdx.1,uc009bdy.1,uc009bdz.1,uc009bea.1,uc009beb.1: | 1.619537972 | 1.38E−05 | 0.0018580 |
| chr6:−:29490826:29559607:Tnpo3:6: | | | |
| uc009iqx.1,uc009iqy.1,uc009iqz.1,uc009ira.1,uc009irb.1,uc009irc.1, | 1.265715433 | 1.46E−05 | 0.0018925 |
| uc012fqw.1:chr7:−:109267913:109358634:Nup98:<1-3-4-5: | | | |
| uc007exv.1:chr10:+:41239305:41250848:Cd164:<1-2:4 | 2.182366474 | 1.65E−05 | 0.0019004 |
| uc009bke.2,uc009bkf.1,uc009bkg.1,uc009bkh.2,uc012ekc.1: | 2.384371493 | 1.88E−05 | 0.0020863 |
| chr6:+:38383924:38474790:Ubn2,mKIAA2030:<2:4-5-6-8-9-10-11-12-13 | | | |
| uc008tkj.2,uc008tkk.2,uc012dgn.1:chr4:−:82444641:82505565: | 1.468746597 | 2.14E−05 | 0.0022260 |
| Gramp3,Zdhhc21:<2-3-4: | | | |
| uc008dzi.1,uc008dzj.1,uc008dzl.1:chr18:−:6435948:6516085:Epc1:<2: | 1.231453936 | 2.15E−05 | 0.0022260 |
| uc009mrt.2,uc009mru.2,uc009mrv.2,uc009mrw.2,uc009mrx.2, | 1.332571048 | 3.34E−05 | 0.0032470 |
| uc009mry.1:chr8:+:91220926:91275844:CyId::12 | | | |
| uc009ptb.1,uc009ptc.1,uc009ptd.1,uc009pte.1,uc009ptf.1:chr9: | 2.394708794 | 3.72E−05 | 0.0035066 |
| +:56266652:56344743:Hmg20a:<1-2-3-5: | | | |
| uc007jit.1,uc007jiu.1,uc007jiv.1,uc007jiw.1,uc007jix.1,uc007jiy.1, | 1.040256109 | 5.36E−05 | 0.0049005 |
| uc007jiz.1,uc007jja.1,uc007jjb.1,uc007jjc.1,uc007jjd.1:chr11: | | | |
| −:62130191:62271308:Ncor1:10: | | | |
| uc009uhk.1,uc009uhl.2,uc009uhm.1,uc009uhn.1,uc009uho.1, | 1.551835804 | 5.73E−05 | 0.0050868 |
| uc009uhp.1,uc009uhq.1:chrX:+:132277230:132338007:Armcx5, | | | |
| Gprasp1:<1:4 | | | |
| uc009rtg.1,uc009rth.1,uc009rti.1,uc009rtj.1,uc009rtk.1,uc009rtl.1: | 1.595252556 | 6.27E−05 | 0.0054098 |
| chr9:−:109986823:110019918:Dhx30,HELG:<3-5:7-8-10> | | | |
| uc007egu.2,uc007egv.1,uc007egw.1,uc007egx.2,uc011wzo.1, | 1.796772712 | 0.0001014 | 0.0070047 |
| uc011wzp.1,uc011wzq.1,uc011wzr.1:chr10:−:5342779:5734495: | | | |
| Esr1:<8-9: | | | |
| uc008kia.1,uc008kib.1,uc008kic.1,uc008kid.1:chr2:+:83564553: | 1.039353216 | 0.0001015 | 0.0070047 |
| 83647073:Itgav:<3-5: | | | |
| uc009lom.1,uc009lon.2,uc009loo.1,uc012gdb.1:chr8:+:46035561: | 1.367947818 | 0.0001048 | 0.0070761 |
| 46137611:Fat1,mfat1:<8-9-10-11-12-13-14-15-16-17-18- | | | |
| 19-20-21-22-23-24-25:29> | | | |
| uc008fww.2,uc008fwy.2,uc008fwz.2,uc008fxa.2,uc008fxb.2, | 1.515675889 | 0.0001158 | 0.0074933 |
| uc008fxc.2,uc012bfy.1:chr19:+:3767420:3818303:Suv420h1: | | | |
| 7-8:10-11-12 | | | |
| uc008pbm.1,uc008pbn.1,uc008pbo.1,uc012cpb.1,uc012cpc.1: | 1.705613624 | 0.0001259 | 0.0075249 |
| chr3:+:40603872:40620805:Plk4:<2-3-4-5-6: | | | |
| uc008qzr.3,uc008qzs.1,uc008qzt.3,uc012cwf.1:chr3:−:108596097: | 1.062012217 | 0.0001342 | 0.0077204 |
| 108643420:Stxbp3a:<1-2-3-4: | | | |
| uc008dfn.1,uc008dfo.2,uc008dfp.1,uc008dfq.2,uc008dfr.2,uc008dfs.2: | 1.105627077 | 0.0001373 | 0.0077539 |
| chr17:+:64213329:64488845:Fer,Fert2:<3-4-5-6-7- | | | |
| 8-9-11-14:16-17-18> | | | |
| uc009tvc.1,uc009tvd.1,uc009tve.1,uc009tvf.1:chrX:+:96131654: | 1.510222019 | 0.0001437 | 0.0078328 |
| 96144359:Yipf6::12-13> | | | |
| uc008qca.1,uc008qcb.1,uc012csw.1:chr3:+:90097105:90162042: | −2.534665066 | 0.0001471 | 0.0078793 |
| Gatad2b:<3-4:6-7-8-9-11-12-13-14> | | | |
| uc009rtm.1,uc009rtn.1,uc009rto.1,uc009rtp.1,uc012hbg.1:chr9: | 1.698661263 | 0.000153 | 0.0080560 |
| +:110034527:110142682:Smarcc1:17-18-19-20-21-22-23-24-25-26: | | | |
| uc008jzt.1,uc008jzu.1:chr2:−:70550464:70663537:T1k1,mKIAA0137:: | −1.374730407 | 0.0001864 | 0.0096543 |
| 5-6-7-8-9-10-11-12-13-15-16-17-18-19-20-21-22> | | | |
| uc007uvx.2,uc007uvy.2,uc007uvz.1,uc007uwa.1,uc007uwb.2, | 1.648767329 | 0.0002016 | 0.0102345 |
| uc007uwc.1,uc007uwd.1,uc011zpj.1:chr14:+:102129144:102333910: | | | |
| Lmo7::12-18-19-20-21-22-23 | | | |
| uc007ivf.2:chr11:+:52045496:52060360:Skp1a:3:5-6> | −1.493660612 | 0.0002042 | 0.0102345 |
| uc007zuj.2,uc007zuk.2,uc012ahp.1:chr16:+:87455229:87483758: | 1.292429748 | 0.0002204 | 0.0108718 |
| Usp16:4-5-6-7: | | | |
| uc008szw.2,uc008szx.2:chr4:−:59484739:59562236:Rod1:<2- | −1.535280346 | 0.0002249 | 0.0109192 |
| 3:5-6-7-8-9-10-11-12-13-14 | | | |
| uc007hag.2,uc007hah.2,uc007hai.2:chr10:+:111409750:111425486: | 1.720441914 | 0.0002678 | 0.0128008 |
| Krr1:<2-4-5: | | | |
| uc007vep.1,uc007veq.1,uc007ver.1,uc007ves.1,uc007vet.1,uc007veu.1, | −1.104466332 | 0.000275 | 0.0128411 |
| uc007vev.1:chr15:−:8241224:8415285:Nipbl:49-50-51-52:55> | | | |
| uc007hgm.1,uc007hgn.1,uc007hgo.1,uc007hgp.1,uc007hgq.1, | −1.442152892 | 0.0002809 | 0.0128411 |
| uc007hgr.1,uc007hgs.1,uc007hgt.1,uc011xpf.1:chr10:−:122550299: | | | |
| 122633979:Usp15:<1-2-3-4-5-6:11-12-13-14-16-17-19- | | | |
| 20-21-22-23-25-26-28-29> | | | |
| uc009odn.1,uc009odo.1:chr9:+:8899832:8968611:Pgr<2-3-4: | 1.155311256 | 0.0002844 | 0.0128411 |
| uc007rxm.2:chr13:−:115078002:115178302:Ndufs4:<1:3 | −2.290814963 | 0.0003031 | 0.0132384 |
| uc008sbo.1,uc008sbp.1,uc008sbq.1:chr4:−:15924267:15941024: | 2.343266037 | 0.0003068 | 0.0132384 |
| Osgin2:<2-3-4: | | | |
| uc008ovl.2,uc008ovm.2,uc008ovo.2,uc008ovp.2,uc008ovq.1, | −1.540505304 | 0.0003529 | 0.0148175 |
| uc008ovr.2,uc012cog.1,uc012coh.1:chr3:−:30798216:30868337:Phc3:17: | | | |
| uc008vbx.1,uc008vby.1:chr4:−:132409978:132440373:Stx12:<3-4-5: | −1.666570338 | 0.0003673 | 0.0152158 |
| uc007hnb.1,uc007hnc.1,uc007hnd.1:chr10:−:127927916:127930881: | −1.121674212 | 0.0003919 | 0.0156535 |
| Myl6:<2-3-4-5:7 | | | |
| uc008kxh.2,uc008kxi.2,uc008kxj.2,uc008kxk.1,uc008kxl.2:chr2: | 1.185897573 | 0.0004031 | 0.0158530 |
| +:92024338:92204823:Phf21a:<4-5-6-7-8-9:11 | | | |
| uc008dmp.2,uc008dmq.2,uc012awt.1,uc012awu.1:chr17:−:71906366: | 1.302818624 | 0.0004138 | 0.0159829 |
| 71948101:Trmt61b:<2-3:5> | | | |
| uc007idk.1,uc007idl.1,uc007idm.1,uc007idn.1:chr11:+:20991326: | 1.358153365 | 0.0004218 | 0.0159829 |
| 21050330:Peli1:7: | | | |
| uc007vep.1,uc007veq.1,uc007ver.1,uc007ves.1,uc007vet.1,uc007veu.1, | −1.078848224 | 0.0004767 | 0.0176306 |
| uc007vev.1:chr15:−:8241224:8415285:Nipbl:53: | | | |
| uc009jxn.1,uc009jxo.1,uc009jxp.1,uc009jxq.1,uc009jxr.1,uc009jxs.1: | 1.147953956 | 0.0005275 | 0.0190565 |
| chr7:+:135110992:135125545:AK134717,Fus,Gm10167:<14-15: | | | |
| uc008fqn.1,uc008fqo.1:chr18:+:76401578:76465385:Smad2: | 1.207325358 | 0.0005923 | 0.0206772 |
| <2:4-5-6-7-8-9-10-11> | | | |
| uc008hit.1,uc008hiu.1,uc012blf.1:chr19:−:37973525:38118067: | 1.131386909 | 0.0005996 | 0.0207005 |
| Myof,mKIAA1207:<1-2-3-4-5-6-7-8-9-10-11-12-13-14-15- | | | |
| 16:18-19-20-21-22-23-24-25-26-27-28-29-30-31-32-33-34-35- | | | |
| 36-37-38> | | | |
| uc008uwf.1,uc008uwg.1,uc008uwh.1,uc008uwi.1,uc008uwj.1: | −1.945973157 | 0.0006569 | 0.0222408 |
| chr4:−:128828068:128866870:S100pbp:9-10-11: | | | |
| uc007nxa.1,uc007nxb.1,uc007nxc.1:chr12:+:76409299:76502442: | 1.705829754 | 0.0006713 | 0.0224273 |
| Rhoj:5: | | | |
| uc008imk.1,uc008imm.1,uc008imn.1,uc008imo.1,uc008imp.1, | 1.085304093 | 0.0007211 | 0.0235032 |
| uc008imq.1,uc008imr.1,uc008ims.1,uc008imt.1,uc008imu.1, | | | |
| uc008imv.1,uc008imw.1,uc012brk.1:chr2:+:19831406:20732162: | | | |
| AK018352,Etl4,Skt,mKIAA1217:30: | | | |
| uc007inq.1,uc007inr.1:chr11:+:45665465:45724127:Clint1::3- | −1.483567458 | 0.0007262 | 0.0235032 |
| 4-5-6-7-8-9-10-11 | | | |
| uc008qbt.2,uc008qbu.2,uc008qbv.2,uc008qbw.2:chr3:+:90058202: | −1.686124797 | 0.0007805 | 0.0247522 |
| 90068047:Crtc2::5> | | | |
| uc007pik.1,uc007pil.1,uc007pim.1,uc011yvv.1:chr12:−:120401275: | 1.932925056 | 0.0007807 | 0.0247522 |
| 120476749:Itgb8:<1-2-3: | | | |
| uc007mau.1,uc007mav.1,uc007maw.1,uc007max.1:chr11:+: | 1.847242778 | 0.0008073 | 0.0252449 |
| 107409273:107548257:Helz:<2:4-5-6-7-8-9-10-11-12-13-14-15-16> | | | |
| uc007uim.1,uc007uin.1,uc007uio.1:chr14:+:65271367:65425133: | −1.453319444 | 0.0008125 | 0.0252449 |
| Kif13b,mKIAA0639::37> | | | |
| uc008fww.2,uc008fwy.2,uc008fwz.2,uc008fxa.2,uc008fxb.2, | 2.159104251 | 0.0008426 | 0.0259194 |
| uc008fxc.2,uc012bfy.1:chr19:+:3767420:3818303:Suv420h1:14:18> | | | |
| uc008xnw.1,uc008xnx.1,uc008xny.1:chr5:−:66006498:66089095: | 1.748492629 | 0.0008725 | 0.0265772 |
| Pds5a:25: | | | |
| uc008dgs.1,uc008dgt.1,uc008dgu.1,uc008dgv.1:chr17:−:66316840: | 1.38109482 | 0.0009092 | 0.0271622 |
| 66426386:Ankrd12:<8-9: | | | |
| uc007dgz.1,uc007dha.1,uc007dhb.1,uc007dhc.1:chr1:−:164805177: | 1.938494602 | 0.0009392 | 0.0275290 |
| 164828843:Fmo2:<2-3-4: | | | |
| uc007woc.1,uc007wod.1,uc007woe.1,uc011zyk.1:chr15:−:77591018: | −1.597541902 | 0.0009548 | 0.0277260 |
| 77672545:Myh9:5-6-7-8-9-10-11:13 | | | |
| uc009nvl.1,uc009nvm.1,uc009nvn.1,uc009nvo.1,uc009nvp.1, | 1.04696396 | 0.0011157 | 0.0309521 |
| uc009nvq.1,uc009nvr.1,uc012gmr.1:chr8:+:125897652:125928073: | | | |
| Tcf25,mKIAA1049::21-22 | | | |
| uc009jds.1,uc009jdt.1,uc009jdu.1,uc009jdv.1,uc009jdw.1:chr7: | 2.000224414 | 0.0012001 | 0.0323152 |
| −:116667424:116760661:St5::3-4-5-7-8> | | | |
| uc007zjb.2,uc007zjc.1,uc012agi.1,uc012agj.1:chr16:−:45746345: | 1.311774431 | 0.0012562 | 0.0330305 |
| 45953711:Phldb2:<5-6-7-8-9-10-11:15-16-17-18-19-20-21-22> | | | |
| uc008awq.2,uc008awr.2,uc008aws.2,uc012aml.1:chr17:−:24645794: | −1.135405811 | 0.001278 | 0.0330305 |
| 24664883:Traf7:<1-2-4:6-8> | | | |
| uc007vdw.2,uc007vdx.2,uc007vdy.2,uc007vdz.2:chr15:+:7079571: | 1.184726082 | 0.0012847 | 0.0330305 |
| 7147489:Lifr:<4-5-6-7-8-9-10-11-12-13-14-15-16-17: | | | |
| uc009ltr.1,uc009lts.1,uc009ltt.1,uc009ltu.1,uc009ltv.1,uc012ged.1, | −1.382815991 | 0.0012864 | 0.0330305 |
| uc012gee.1,uc012gef.1:chr8:+:63472016:63609031:Nek1:21: | | | |
| uc007dgo.1,uc007dgp.1,uc007dgq.1,uc007dgr.1,uc007dgs.1, | 1.42081838 | 0.0013334 | 0.0339576 |
| uc007dgt.1,uc007dgu.1:chr1:−:164601915:164670687:Bat2d, | | | |
| Bat2l2,Prrc2c:9:11-12-13-14-15 | | | |
| uc007ouq.1,uc007our.1,uc007ous.1,uc007out.1,uc007ouu.1: | 1.487978373 | 0.0014552 | 0.0361705 |
| chr12:−:104022857:104116616:Btbd7:6: | | | |
| uc008zke.1,uc008zkf.1:chr5:+:122161617:122264959:Atxn2:22-23:25> | 1.581849902 | 0.0015698 | 0.0380419 |
| uc009and.1,uc009ane.1,uc012egz.1:chr5:+:147042825:147114450: | 1.126746007 | 0.0015842 | 0.0380419 |
| Cdk8::11-12-13-14-15-16> | | | |
| uc009afw.1,uc009afx.1:chr5:−:139452928:139470907:Pdgfa::7> | −1.513651741 | 0.0016091 | 0.0381636 |
| uc008ekn.1,uc008eko.1:chr18:−:34602004:34666477:Fam13b::18 | 1.000329063 | 0.0017281 | 0.0400269 |
| uc009ugp.2,uc009ugq.2,uc009ugr.2,uc009ugs.2:chrX:−:131325991: | −2.037048884 | 0.0017363 | 0.0400269 |
| 131343760:Armcx2,mKIAA0512::6-7-8> | | | |
| uc008eeq.1,uc008eer.1,uc008ees.2:chr18:+:20716616:20763027: | 1.16351841 | 0.0017521 | 0.0400269 |
| Dsg2:9: | | | |
| uc009hie.1,uc009hif.1,uc009hig.1,uc009hih.1,uc009hii.1,uc009hij.1, | −1.644131014 | 0.0018318 | 0.0413190 |
| uc009hil.1:chr7:−:74378716:74517744:Mef2a:8:11-12-13-15-16-17> | | | |
| uc008xnw.1,uc008xnx.1,uc008xny.1:chr5:−:66006498:66089095: | −1.180164113 | 0.0018408 | 0.0413190 |
| Pds5a::25 | | | |
| uc008sey.1,uc008sez.1,uc008sfa.1,uc008sfb.1,uc008sfc.1,uc008sfd.1: | 1.685050611 | 0.0018485 | 0.0413190 |
| chr4:+:32744093:32862192:AK037999,Mdn1:<5-6- | | | |
| 7-8-9-10-11-12-13-14-15-16-17-18-19-20-21-22-23-24-25-26-27: | | | |
| uc007eeu.1,uc007eev.1,uc007eew.1,uc007eex.1:chr1:−:196923575: | −1.29892634 | 0.0020387 | 0.0449248 |
| 196957764:Cr1l,Crry:12: | | | |
| uc008gpw.2,uc008gpx.2,uc008gpy.2,uc008gpz.2,uc008gqa.2: | 1.23093707 | 0.002126 | 0.0461923 |
| chr19:+:10599733:10622225:Cpsf7:<2-3-5-6:8-9-11-12 | | | |

TABLE 5
DEXSeq

| geneID | exonID | dispersion | pvalue | padjust | meanBase | log2fold(R/G) |
|---|---|---|---|---|---|---|
| uc012gcj.1 + uc009llr.2 + uc009llv.1 + uc009lls.2 + uc009llu.1 + uc009l | E021 | 0.175052 | 2.22E−16 | 3.38E−11 | 41.53023 | 2.620127 |
| lq.2 + uc009llt.1 | | | | | | |
| uc007gwx.2 + uc007gwy.2 | E002 | 0.07751 | 1.07E−13 | 5.43E−09 | 768.9806 | 0.731335 |
| uc008jjn.2 | E040 | 0.100835 | 9.68E−14 | 5.43E−09 | 148.2038 | 14.29697 |
| uc008sys.1 + uc008syr.1 + uc008syt.1 + uc008syu.2 | E011 | 0.265173 | 6.28E−09 | 0.000239 | 193.1942 | −1.03589 |
| uc007zmq.2 + uc007zmp.2 + uc007zmu.2 + uc012agr.1 + uc007zmn. | E028 | 0.184129 | 2.54E−08 | 0.000775 | 38.17011 | 1.675024 |
| 2 + uc007zms.2 + uc007zmr.2 + uc007zmt.2 + uc007zmo.2 | | | | | | |
| uc007zmq.2 + uc007zmp.2 + uc007zmu.2 + uc012agr.1 + uc007zmn. | E027 | 0.191135 | 3.46E−08 | 0.000879 | 35.92626 | 1.75157 |
| 2 + uc007zms.2 + uc007zmr.2 + uc007zmt.2 + uc007zmo.2 | | | | | | |
| uc009sqe.2 + uc009sqj.2 + uc009sqd.1 + uc012het.1 + uc009sqh.2 + u | E002 | 0.180508 | 4.04E−08 | 0.00088 | 39.44305 | 1.457519 |
| c009sqg.2 + uc009sqi.2 + uc012heu.1 + uc009sqc.1 + uc009sqf.2 | | | | | | |
| uc008hvd.1 | E002 | 0.489596 | 7.16E−08 | 0.001121 | 10.25295 | 14.8611 |
| uc008hvd.1 | E003 | 0.105208 | 7.16E−08 | 0.001121 | 128.725 | −0.00089 |
| uc008xua.1 + uc008xud.1 + uc008xue.1 + uc008xuc.1 + uc008xuf.1 + | E028 | 0.073395 | 7.81E−08 | 0.001121 | 2945.284 | −0.35369 |
| uc008xub.1 | | | | | | |
| uc009oow.1 | E015 | 0.109534 | 8.09E−08 | 0.001121 | 113.9115 | −0.6584 |
| uc008ym1.2 + uc012eam.1 + uc008ymg.2 + uc008ymi.2 + uc008ymh. | E007 | 0.204804 | 2.00E−07 | 0.002546 | 32.23037 | −1.01081 |
| 2 + uc008ymk.1 + uc008ymj.2 | | | | | | |
| uc008cvt.1 + uc008cvv.1 + uc008cvu.1 + uc008cyr.1 + uc008cvw.1 + u | E007 | 0.121276 | 6.26E−07 | 0.00734 | 86.79955 | −0.73149 |
| c008cvs.1 | | | | | | |
| uc007gub.1 + uc007guc.2 + uc007gtz.2 + uc007gua.1 | E017 | 0.255653 | 1.03E−06 | 0.009304 | 32.13604 | −0.9669 |
| uc007hof.1 + uc007hoh.2 + uc007hog.2 | E011 | 0.239954 | 9.75E−07 | 0.009304 | 40.40718 | −1.22753 |
| uc008pig.1 + uc008pit.2 + uc008pih.1 + uc008pii.2 | E049 | 0.559838 | 9.46E−07 | 0.009304 | 20.99629 | 1.516188 |
| uc009quu.1 + uc009qut.1 + uc009qus.1 | E055 | 0.102101 | 1.04E−06 | 0.009304 | 141.9836 | −0.65819 |
| uc007dsa.1 + uc007dsb.2 + uc007dry.1 + uc007drw.1 + uc007drx.1 | E011 | 0.145119 | 1.14E−06 | 0.009648 | 58.51785 | −0.85658 |
| uc009bug.1 + uc009bui.1 + uc009buh.1 + uc012eld.1 + uc009bue.1 + | E002 | 0.398821 | 1.42E−06 | 0.011394 | 13.10022 | 1.323474 |
| uc009buf.1 + uc009buj.1 | | | | | | |
| uc009sqe.2 + uc009sqj.2 + uc009sqd.1 + uc012het.1 + uc009sqh.2 + u | E006 | 0.204901 | 2.12E−06 | 0.016147 | 133.1533 | 0.98514 |
| c009sqg.2 + uc009sqi.2 + uc012heu.1 + uc009sqc.1 + uc009sqf.2 | | | | | | |
| uc007dhe.1 + uc007dhf.1 | E001 | 0.261366 | 2.24E−06 | 0.016256 | 22.60629 | 1.465804 |
| uc007qwr.1 | E001 | 0.115478 | 2.88E−06 | 0.01776 | 98.35805 | 0.54106 |
| uc007umn.2 + uc007umo.2 | E011 | 0.710656 | 3.03E−06 | 0.01776 | 14.39885 | −1.77866 |
| uc008zge.1 + uc012eck.1 + uc008zgf.1 | E004 | 0.53646 | 3.03E−06 | 0.01776 | 9.218573 | 2.222311 |
| uc011ymc.1 + uc007npj.2 + uc007npk.1 | E001 | 0.216573 | 2.82E−06 | 0.01776 | 29.60757 | −1.45534 |
| uc012gcj.1 + uc009llr.2 + uc009llv.1 + uc009lls.2 + uc009llu.1 + uc009l | E019 | 0.470647 | 2.75E−06 | 0.01776 | 10.74025 | 2.742949 |
| lq.2 + uc009llt.1 | | | | | | |
| uc008sum.1 + uc012ddw.1 | E040 | 0.08047 | 4.37E−06 | 0.024672 | 502.0965 | −0.41889 |
| uc007zmq.2 + uc007zmp.2 + uc007zmu.2 + uc012agr.1 + uc007zmn. | E046 | 0.100914 | 5.93E−06 | 0.032278 | 147.8002 | −0.5011 |
| 2 + uc007zms.2 + uc007zmr.2 + uc007zmt.2 + uc007zmo.2 | | | | | | |
| uc007mdg.2 + uc011ygu.1 + uc007mdf.1 | E019 | 0.121229 | 6.34E−06 | 0.033324 | 114.8881 | −0.72349 |
| uc009dkt.2 + uc009dks.2 + uc009dku.2 | E006 | 0.076659 | 7.36E−06 | 0.037402 | 907.6902 | −0.22362 |
| uc009jhu.2 + uc009jhv.2 | E004 | 0.192424 | 7.62E−06 | 0.037474 | 35.54204 | 13.34002 |
| uc009jhu.2 + uc009jhv.2 | E007 | 0.159964 | 8.60E−06 | 0.040979 | 48.64871 | 2.081461 |
| uc007mah.2 + uc007maj.2 + uc007mai.2 | E001 | 0.354497 | 9.25E−06 | 0.042743 | 23.06571 | 1.490329 |

TABLE 6
MATS SE

| | | gene | | | exonStart_ | exon | upstream | upstream | downstream | downstream |
|---|---|---|---|---|---|---|---|---|---|---|
| ID | GeneID | Symbol | chr | strand | Obase | End | ES | EE | ES | EE |
| 5068 | Q62009-3 | Postn | chr3 | + | 54184333 | 54184414 | 54181482 | 54181528 | 54187309 | 54187399 |
| 239 | Q544R1 | Prosc | chr8 | + | 28155869 | 28155905 | 28155499 | 28155607 | 28156395 | 28156471 |
| 18661 | Q68FH0 | Pkp4 | chr2 | + | 59188550 | 59188679 | 59185980 | 59186098 | 59190318 | 59190392 |

TABLE 7
MATS SE 2

| | IC_ | SC_ | IC_ | SC_ | Inc | Skip | | | | | IncLevel |
|---|---|---|---|---|---|---|---|---|---|---|---|
| | SAM- | SAM- | SAM- | SAM- | Form | Form | | | | | Differ- |
| ID | PLE_1 | PLE_1 | PLE_2 | PLE_2 | Len | Len | PValue | FDR | IncLevel1 | IncLevel2 | ence |
| 5068 | 94, 215, | 93, 158, | 560, 24 | 137, 793, 7 | 100 | 37 | 2.54E−09 | 4.44E−05 | 0.272, 0.33 | 0.131, 0.08 | 0.204 |
| | 104, 165 | 77, 164 | 7, 313, 4 | 15, 391, 39 | | | | | 5, 0.333, 0.2 | 9, 0.07, 0.1 | |
| | | | 48 | 2 | | | | | 71 | 06 | |
| 239 | 9, 6, 2, 7 | 4, 2, 11, 7 | 11, 6, | 1, 1, 0, 4 | 42 | 69 | 1.58E−06 | 0.013767 | 0.787, 0.83 | 0.948, 0.90 | −0.324 |
| | | | 17, 24 | | | | | | 1, 0.23, 0.62 | 8, 1.0, 0.90 | |
| | | | | | | | | | 2 | 8 | |
| 18661 | 3, 22, 0, | 45, 45, 3, | 15, 29, | 11, 2, 8, 9 | 182 | 67 | 3.58E−06 | 0.020812 | 0.024, 0.15 | 0.334, 0.84 | −0.318 |
| | 4 | 14 | 3, 8 | | | | | | 3, 0.0, 0.095 | 2, 0.121, 0. | |
| | | | | | | | | | | 247 | |

TABLE 8
MATS MXE

| | | | | | | | 2nd | | | | | |
|---|---|---|---|---|---|---|---|---|---|---|---|---|
| | | | | | 1stExon | 1st | Exon | 2nd | | | down- | down- |
| | | gene | | | Start_ | Exon | Start_ | Exon | upstream | upstream | stream | stream |
| ID | GeneID | Symbol | chr | strand | Obase | End | Obase | End | ES | EE | ES | EE |
| 1825 | NP_ | Chd2 | chr7 | − | 80638310 | 80638527 | 80642592 | 80642717 | 80636220 | 80636310 | 80644479 | 80644658 |
| | 001074814 | | | | | | | | | | | |
| 899 | uc009ual.2 | 5530601 | chrX | − | 1.02E + 08 | 1.02E+08 | 1.02E + 08 | 1.02E + 08 | 1.02E + 08 | 1.02E+08 | 1.02E+08 | 1.02E+08 |
| | | H04Rik | | | | | | | | | | |
| 32 | Q544R1 | Prosc | chr8 | + | 28156395 | 28156471 | 28159645 | 28159780 | 28155499 | 28155607 | 28161730 | 28161873 |

TABLE 9
MATS MXE2

| | IC_ | SC_ | IC_ | SC_ | Inc | Skip | | | | | IncLevel |
|---|---|---|---|---|---|---|---|---|---|---|---|
| | SAM- | SAM- | SAM- | SAM- | Form | Form | | | | | Differ- |
| ID | PLE_1 | PLE_1 | PLE_2 | PLE_2 | Len | Len | PValue | FDR | IncLevel1 | IncLevel2 | ence |
| 1825 | 81, 36, | 120, 54, | 26, 14, | 136, 50, | 194 | 286 | 2.51E−05 | 0.029963 | 0.499, 0.496, | 0.22, 0.292, | 0.219 |
| | 45, 20 | 42, 29 | 22, 19 | 40, 74 | | | | | 0.612, 0.504 | 0.448, 0.275 | |
| 899 | 13, 7, | 5, 11, 11, | 7, 7, 2, | 12, 16, | 173 | 181 | 1.63E−05 | 0.029963 | 0.731, 0.4, | 0.379, 0.314, | 0.38 |
| | 16, 9 | 0 | 12 | 24, 16 | | | | | 0.603, 1.0 | 0.08, 0.44 | |
| 32 | 4, 2, 11, | 14, 8, 5, 3 | 1, 1, 0, 5 | 8, 10, | 138 | 206 | 5.83E−05 | 0.046399 | 0.299, 0.272, | 0.157, 0.13, | 0.336 |
| | 7 | | | 19, 8 | | | | | 0.767, 0.777 | 0.0, 0.483 | |

Thus, specific compositions and methods of modeling and predicting differential alternative splicing events and applications thereof have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

### REFERENCES:

1. Ingolia, N. T. (2014) “Ribosome Profiling: New Views of Translation, from Single Codons to Genome Scale,” Nat. Rev. Genet. 15(3), 205-213.
2. Weiss, R. B. and Atkins, J. F. (2011) “Translation Goes Global,” Science 334(6062), 1509-1510.
3. Anderson, M. L. M. and Young, B. D. (1985) “Quantitative Filter Hybridization,” in Nucleic Acid Hybridisation: A Practical Approach (Hames, B. D. and Higgins, S. J., Eds.), pp 73-111, Oxford University Press, USA.
4. Dieffenbach, C. W. and Dveksler, G. S. (1995) PCR Primer, a Laboratory Manual, Cold Spring Harbor Laboratory Press, Plainview, N.Y.
5. Mullis, K. B. et al. “Process for Amplifying, Detecting, and/or-Cloning Nucleic Acid Sequences,” U.S. Pat. No. 4,683,195, application Ser. No. 06/828,144, filed Feb. 7, 1986. (issued Jul. 28, 1987).
6. Mullis, K. B. “Process for Amplifying Nucleic Acid Sequences,” U.S. Pat. No. 4,683,202, application Ser. No. 06/791,308, filed Oct. 25, 1985. (issued Jul. 28, 1987).
7 Maniatis, T. et al. (1987) “Regulation of Inducible and Tissue-Specific Gene Expression,” Science 236(4806), 1237-1245.
8. Sambrook, J. et al., (Eds.) (1989) Molecular Cloning: A Laboratory Manual, 2nd ed., Cold Spring Harbor Laboratory Press, New York.
9. Ozsolak, F. and Milos, P. M. (2011) “RNA Sequencing: Advances, Challenges and Opportunities,” Nat. Rev. Genet. 12(2), 87-98.
10. Wilhelm, B. T. and Landry, J.-R. (2009) “RNA-Seq—Quantitative Measurement of Expression through Massively Parallel RNA-Sequencing,” Methods 48(3), 249-257.
11. De Wit, P. et al. (2012) “The Simple Fool's Guide to Population Genomics Via RNA-Seq: An Introduction to High-Throughput Sequencing Data Analysis,” Mol. Ecol. Resour. 12(6), 1058-1067.
12. Ingolia, N. T. et al. (2012) “The Ribosome Profiling Strategy for Monitoring Translation in vivo by Deep Sequencing of Ribosome-Protected mRNA Fragments,” Nat. Protoc. 7(8), 1534-1550.
13. Stuber, G. D. and Mason, A. O. (2013) “Integrating Optogenetic and Pharmacological Approaches to Study Neural Circuit Function: Current Applications and Future Directions,” Pharmacol. Rev. 65(1), 156-170.
14. Eswaran, J. et al. (2013) “RNA Sequencing of Cancer Reveals Novel Splicing Alterations,” Sci. Rep. 3, 1689.
15. Sasaki, T. et al. (2010) “Knockdown of Akt Isoforms by RNA Silencing Suppresses the Growth of Human Prostate Cancer Cells in vitro and in vivo,” Biochem. Biophys. Res. Commun. 399(1), 79-83.
16. Bhardwaj, A. R. et al. (2015) “Global Insights into High Temperature and Drought Stress Regulated Genes by RNA-Seq in Economically Important Oilseed Crop Brassica Juncea,” BMC Plant Biol. 15, 9.
17. Cheng, A. W. et al. (2014) “Muscleblind-Like 1 (Mbnl1) Regulates Pre-mRNA Alternative Splicing During Terminal Erythropoiesis,” Blood 124(4), 598-610.
18. Wittrup, A. and Lieberman, J. (2015) “Knocking Down Disease: A Progress Report on siRNA Therapeutics,” Nat. Rev. Genet. 16(9), 543-552.
19. Ingolia, N. T. et al. (2009) “Genome-Wide Analysis in Vivo of Translation with Nucleotide Resolution Using Ribosome Profiling,” Science 324(5924), 218-223.
20. Black, D. L. (2003) “Mechanisms of Alternative Pre-Messenger RNA Splicing,” Annu. Rev. Biochem. 72, 291-336.
21. Wang, E. T. et al. (2008) “Alternative Isoform Regulation in Human Tissue Transcriptomes,” Nature 456(7221), 470-476.
22. Pan, Q. et al. (2008) “Deep Surveying of Alternative Splicing Complexity in the Human Transcriptome by High-Throughput Sequencing,” Nat Genet 40(12), 1413-1415.
23. Ranum, L. P. W. and Cooper, T. A. (2006) “RNA-Mediated Neuromuscular Disorders,” Annu. Rev. Neurosci. 29, 259-277.
24. Kuyumcu-Martinez, N. M. and Cooper, T. A. (2006) “Misregulation of Alternative Splicing Causes Pathogenesis in Myotonic Dystrophy,” Prog. Mol. SubCell Biol 44, 133-159.
25. Mordes, D. et al. (2006) “Pre-mRNA Splicing and Retinitis Pigmentosa,” Mol. Vis. 12, 1259-1271.
26. Grosso, A. R. et al. (2008) “The Emerging Role of Splicing Factors in Cancer,” EMBO Rep. 9(11), 1087-1093.
27. Sammeth, M. et al. (2008) “A General Definition and Nomenclature for Alternative Splicing Events,” PLoS Comput. Biol. 4(8), e1000147.
28. Katz, Y. et al. (2010) “Analysis and Design of RNA Sequencing Experiments for Identifying Isoform Regulation,” Nat. Meth. 7(12), 1009-1015.
29. Shen, S. et al. (2012) “Mats: A Bayesian Framework for Flexible Detection of Differential Alternative Splicing from RNA-Seq Data,” Nucleic Acids Res. 40(8), e61.
30. Campbell, G. (2010) “Guidance for the Use of Bayesian Statistics in Medical Device Clinical Trials,” (Services, U. S. D. o. H. a. H., Ed.).
31. Anders, S. et al. (2012) “Detecting Differential Usage of Exons from RNA-Seq Data,” Genome Res. 22(10), 2008-2017.
32. Trapnell, C. et al. (2010) “Transcript Assembly and Quantification by RNA-Seq Reveals Unannotated Transcripts and Isoform Switching During Cell Differentiation,” Nat. Biotechnol. 28(5), 511-515.
33. Howard, B. E. et al. (2013) “High-Throughput RNA Sequencing of Pseudomonas-Infected Arabidopsis Reveals Hidden Transcriptome Complexity and Novel Splice Variants,” PLoS. ONE 8(10), e74183.
34. Yu, P. and Shaw, C. A. (2014) “An Efficient Algorithm for Accurate Computation of the Dirichlet-Multinomial Log-Likelihood Function,” Bioinformatics 30(11), 1547-1554.
35. Visbal, A. P. et al. (2011) “Altered Differentiation and Paracrine Stimulation of Mammary Epithelial Cell Proliferation by Conditionally Activated Smoothened,” Dev. Biol. 352(1), 116-127.
36. Moraes, R. C. et al. (2007) “Constitutive Activation of Smoothened (SMO) in Mammary Glands of Transgenic Mice Leads to Increased Proliferation, Altered Differentiation and Ductal Dysplasia,” Development 134(6), 1231-1242.
37. Dobin, A. et al. (2013) “Star: Ultrafast Universal RNA-Seq Aligner,” Bioinformatics 29(1), 15-21.
38. Casella, G. and Berger, R. L. (2001) Statistical Inference, 2 ed., Thomson Learning.
39. Anders, S. and Huber, W. (2010) “Differential Expression Analysis for Sequence Count Data,” Genome Biol. 11(10), R106.
40. Cho, V. et al. (2014) “The RNA-Binding Protein Hnrnpll Induces a T Cell Alternative Splicing Program Delineated by Differential Intron Retention in Polyadenylated RNA,” Genome Biol. 15(1), R26.
41. Trapnell, C. et al. (2012) “Differential Gene and Transcript Expression Analysis of RNA-Seq Experiments with Tophat and Cufflinks,” Nat. Protoc. 7(3), 562-578.
42. Hoersch, S. and Andrade-Navarro, M. A. (2010) “Periostin Shows Increased Evolutionary Plasticity in Its Alternatively Spliced Region,” BMC Evol. Biol. 10, 30.
43. Kim, C. J. et al. (2008) “Role of Alternative Splicing of Periostin in Human Bladder Carcinogenesis,” Int. J. Oncol. 32(1), 161-169.
44. Contie, S. et al. (2010) “Development of a New Elisa for Serum Periostin: Evaluation of Growth-Related Changes and Bisphosphonate Treatment in Mice,” Calcif. Tissue Int. 87(4), 341-350.
45. Sasaki, H. et al. (2003) “Elevated Serum Periostin Levels in Patients with Bone Metastases from Breast but Not Lung Cancer,” Breast Cancer Res. Treat. 77(3), 245-252.
46. Ma, X. J. et al. (2009) “Gene Expression Profiling of the Tumor Microenvironment During Breast Cancer Progression,” Breast Cancer Res. 11(1), R7.
47. Rustici, G. et al. (2013) “Arrayexpress Update—Trends in Database Growth and Links to Data Analysis Tools,” Nucleic Acids Res. 41(Database issue), D987-990.
48. McCarthy, D. J. et al. (2012) “Differential Expression Analysis of Multifactor RNA-Seq Experiments with Respect to Biological Variation,” Nucleic Acids Res. 40(10), 4288-4297.
49. McCullagh, P. and Nelder, J. A. (1989) Generalized Linear Models, Chapman and Hall/CRC.
50. Wu, J. et al. (2011) “Splicetrap: A Method to Quantify Alternative Splicing under Single Cellular Conditions,” Bioinformatics 27(21), 3010-3016.
51. Li, H. et al. (2009) “The Sequence Alignment/Map Format and Samtools,” Bioinformatics 25(16), 2078-2079.
52. Li, G. et al. (2002) “Conditional Loss of Pten Leads to Precocious Development and Neoplasia in the Mammary Gland,” Development 129(17), 4159-4170.
53. Welm, B. E. et al. (2008) “Lentiviral Transduction of Mammary Stem Cells for Analysis of Gene Function During Development and Cancer,” Cell Stem Cell 2(1), 90-102.
54. LaMarca, H. L. et al. (2010) “Ccaat/Enhancer Binding Protein Beta Regulates Stem Cell Activity and Specifies Luminal Cell Fate in the Mammary Gland,” Stem Cells 28(3), 535-544.
55. Anders, S. et al. (2014) “Htseq—a Python Framework to Work with High-Throughput Sequencing Data,” Bioinformatics 31(2), 166-169.
56. Hsu, F. et al. (2006) “The Ucsc Known Genes,” Bioinformatics 22(9), 1036-1046.
57. Cormen, T. H. et al. (2009) Introduction to Algorithms, 3 ed., The MIT Press.
58. Sugnet, C. W. et al. (2004) “Transcriptome and Genome Conservation of Alternative Splicing Events in Humans and Mice,” Pac. Symp. Biocomput., 66-77.
59. Skiena, S. S. (2010) The Algorithm Design Manual 2ed., Springer.
60. Agresti, A. (2002) Categorical Data Analysis, Wiley-Interscience.
61. Cameron, C. A. and Trivedi, P. K. (2013) Regression Analysis of Count Data, Cambridge University Press, Cambridge, United Kingdom.
62. Robinson, M. D. et al. (2010) “Edger: A Bioconductor Package for Differential Expression Analysis of Digital Gene Expression Data,” Bioinformatics 26(1), 139-140.
63. Mosimann, J. E. (1962) “On the Compound Multinomial Distribution, the Multivariate Beta-Distribution, and Correlations among Proportions,” Biometrika 49(1/2), 65-82.
64. Hilbe, J. M. (2011) Negative Binomial Regression, 2 ed., Cambridge University Press.
65. Bishop, C. M. (2006) Pattern Recognition and Machine Learning, Springer.
66. Mosimann, J. E. (1962) “On the Compound Multinomial Distribution, the Multivariate $¥Beta$-Distribution, and Correlations among Proportions,” Biometrika 49(1/2), 65-82.
67. Benjamini, Y. and Hochberg, Y. (1995) “Controlling the False Discovery Rate: A Practical and Powerful Approach to Multiple Testing,” Journal of the Royal Statistical Society. Series B (Methodological) 57(1), 289-300.

We claim:

1. A method for predicting differential alternative splicing events from RNA comprising:
a) sequencing at least one said RNA sample to produce RNA sequence data reads per sample;
b) generating one or more directed acyclic graphs from the RNA sequence reads, wherein each directed acyclic graph represents at least a portion of a gene model;
c) extracting count data from the directed acyclic graphs; and
d) generating differential alternative splicing information from the count data using a Dirichlet multinomial (DMN) regression.

2. The method of claim 1, wherein generating one or more directed acyclic graphs in step b) further comprises: Decomposing each directed acyclic graph into alternative splicing types;
and Summarizing the count data into a count table for each decomposed directed acyclic graph.

3. The method of claim 1, further comprising: Aligning the RNA sequence reads to a reference genome or known transcriptome; and Quantifying exon and exon-exon junction reads from the RNA sequence reads.

4. The method of claim 1, wherein the two or more samples are generated under two or more conditions in a multi-factorial experimental design.

5. A method for predicting differential alternative splicing events from ribonucleic acid (RNA) sequence data, comprising:
a) sequencing at least one said RNA sample to produce RNA sequence data reads per sample;
b) generating one or more directed acyclic graphs from the RNA sequence reads, wherein each directed acyclic graph represents at least a portion of a gene model;
c) extracting count data from the directed acyclic graphs;
d) generating differential alternative splicing information from the count data using a Dirichlet multinomial (DMN) regression; and
e) using said alternative splicing information to create antisense RNA sequence which correspond to said alternative splicing information.

6. A method for treating a subject with a disease comprising:
a) sequencing at least one said RNA sample from at least one diseased tissue and at least one control sample to produce RNA sequence data reads per sample;
b) generating one or more directed acyclic graphs from the RNA sequence reads, wherein each directed acyclic graph represents at least a portion of a gene model;
c) extracting count data from the directed acyclic graphs;
d) generating differential alternative splicing information from the count data using a Dirichlet multinomial (DMN) regression;
e) using said alternative splicing information to create antisense RNA sequences which correspond to said alterative splicing information relevant to said diseased tissue sample; and
f) treating said subject with said antisense RNA sequence corresponding to said diseased tissue sample RNA sequence variants so as to alieviate at least one symptom of said disease.

7. A method for predicting differential alternative splicing events from ribonucleic acid (RNA) sequence data, comprising: Receiving RNA sequence reads for two or more samples; Generating one or more directed acyclic graphs from the RNA sequence reads, wherein each directed acyclic graph represents at least a portion of a gene model; Extracting count data from the directed acyclic graphs; and Generating differential alternative splicing information from the count data using a Dirichlet multinomial (DMN) regression.

8. The method of claim 7, wherein generating one or more directed acyclic graphs further comprises: Decomposing each directed acyclic graph into alternative splicing types; and Summarizing the count data into a count table for each decomposed directed acyclic graph.

9. The method of claim 7, further comprising: Aligning the RNA sequence reads to a reference genome or known transcriptome; and Quantifying exon and exon-exon junction reads from the RNA sequence reads.

10. The method of claim 7, wherein the two or more samples are generated under two or more conditions in a multi-factorial experimental design.


SEQUENCE LISTING (48 sequences)

SEQ ID NO: 1 - DNA, 20 bp, Artificial sequence
  Synthetic
cactccaattggttgtccag

SEQ ID NO: 2 - DNA, 20 bp, Artificial sequence
  Synthetic
tggagaacagagggcgacta

SEQ ID NO: 3 - DNA, 22 bp, Artificial sequence
  Synthetic
tgaacttcaaccttttggtttg

SEQ ID NO: 4 - DNA, 20 bp, Artificial sequence
  Synthetic
tgtgcaagtggatgggacta

SEQ ID NO: 5 - DNA, 20 bp, Artificial sequence
  Synthetic
gatccttctgcaccccatac

SEQ ID NO: 6 - DNA, 21 bp, Artificial sequence
  Synthetic
aaggctttggaggaaagtttg

SEQ ID NO: 7 - DNA, 20 bp, Artificial sequence
  Synthetic
tggcaaatacccggattaaa

SEQ ID NO: 8 - DNA, 20 bp, Artificial sequence
  Synthetic
gcaggagacccagaatctca

SEQ ID NO: 9 - DNA, 20 bp, Artificial sequence
  Synthetic
ggattatgccagaagcctca

SEQ ID NO: 10 - DNA, 21 bp, Artificial sequence
  Synthetic
tctggattcatagcctgcttt

SEQ ID NO: 11 - DNA, 22 bp, Artificial sequence
  Synthetic
tgagctacctggtccttactct

SEQ ID NO: 12 - DNA, 20 bp, Artificial sequence
  Synthetic
atccgaacgacatcgagaag

SEQ ID NO: 13 - DNA, 20 bp, Artificial sequence
  Synthetic
agtaacaccaccagcactcg

SEQ ID NO: 14 - DNA, 20 bp, Artificial sequence
  Synthetic
tttgtcttcgcagctgacac

SEQ ID NO: 15 - DNA, 20 bp, Artificial sequence
  Synthetic
ctgcaggagggctacaattt

SEQ ID NO: 16 - DNA, 20 bp, Artificial sequence
  Synthetic
ccaaagcagacaactgctga

SEQ ID NO: 17 - DNA, 20 bp, Artificial sequence
  Synthetic
aggagatcacacagcccttg

SEQ ID NO: 18 - DNA, 20 bp, Artificial sequence
  Synthetic
agagggagagtcagccatca

SEQ ID NO: 19 - DNA, 20 bp, Artificial sequence
  Synthetic
atccgtcgctacgatgattc

SEQ ID NO: 20 - DNA, 20 bp, Artificial sequence
  Synthetic
gaaacaaggtgtggcggtat

SEQ ID NO: 21 - DNA, 20 bp, Artificial sequence
  Synthetic
tagggtagcggctaaggtca

SEQ ID NO: 22 - DNA, 20 bp, Artificial sequence
  Synthetic
cgagcgtccatttcttcttc

SEQ ID NO: 23 - DNA, 20 bp, Artificial sequence
  Synthetic
atgaggccacagagagcaat

SEQ ID NO: 24 - DNA, 20 bp, Artificial sequence
  Synthetic
acacctgtgtccacgctaga

SEQ ID NO: 25 - DNA, 20 bp, Artificial sequence
  Synthetic
tgttctcttgctggtgagga

SEQ ID NO: 26 - DNA, 20 bp, Artificial sequence
  Synthetic
ggcagcaccttcaaagaaat

SEQ ID NO: 27 - DNA, 20 bp, Artificial sequence
  Synthetic
tcacttctgtcaccgtttcg

SEQ ID NO: 28 - DNA, 18 bp, Artificial sequence
  Synthetic
acctcccagccatccaac

SEQ ID NO: 29 - DNA, 21 bp, Artificial sequence
  Synthetic
ccatcagtttgttgacgtttt

SEQ ID NO: 30 - DNA, 20 bp, Artificial sequence
  Synthetic
caaaacctgcagacatggtg

SEQ ID NO: 31 - DNA, 20 bp, Artificial sequence
  Synthetic
ggtcatgagacccacgaact

SEQ ID NO: 32 - DNA, 20 bp, Artificial sequence
  Synthetic
ttgcagctgggaatgctaca

SEQ ID NO: 33 - DNA, 21 bp, Artificial sequence
  Synthetic
aatccatgtgaatccccttgt

SEQ ID NO: 34 - DNA, 24 bp, Artificial sequence
  Synthetic
tcatgtttactttttgcttgtttg

SEQ ID NO: 35 - DNA, 20 bp, Artificial sequence
  Synthetic
tcctgtttgcttccatcaga

SEQ ID NO: 36 - DNA, 20 bp, Artificial sequence
  Synthetic
ccttggtcctcacctctcac

SEQ ID NO: 37 - DNA, 20 bp, Artificial sequence
  Synthetic
ccaaattcgaggagtggtgt

SEQ ID NO: 38 - DNA, 21 bp, Artificial sequence
  Synthetic
aatggcaatagtgtcgtcctg

SEQ ID NO: 39 - DNA, 20 bp, Artificial sequence
  Synthetic
aaccacttctgcagccattc

SEQ ID NO: 40 - DNA, 20 bp, Artificial sequence
  Synthetic
gagctgacttgaggctggag

SEQ ID NO: 41 - DNA, 20 bp, Artificial sequence
  Synthetic
acacacctcagcctggctac

SEQ ID NO: 42 - DNA, 21 bp, Artificial sequence
  Synthetic
ctctgctgcctcctggcttct

SEQ ID NO: 43 - DNA, 21 bp, Artificial sequence
  Synthetic
cgaggcggatcacaagcaata

SEQ ID NO: 44 - DNA, 19 bp, Artificial sequence
  Synthetic
tcaatgggcgggggtcgtt

SEQ ID NO: 45 - DNA, 20 bp, Artificial sequence
  Synthetic
tggtctccaacccattctgc

SEQ ID NO: 46 - DNA, 22 bp, Artificial sequence
  Synthetic
gaacttgtggccgtttacgtcg

SEQ ID NO: 47 - DNA, 20 bp, Artificial sequence
  Synthetic
aaacgttgaatccgaaaaga

SEQ ID NO: 48 - DNA, 20 bp, Artificial sequence
  Synthetic
atccagcttacggatatagt